(19) United States
(12) Patent Application Publication
(10) Pub. No.: US 2010/0112026 A1
(43) Pub. Date: May 6, 2010

(54) SURFACES, METHODS AND DEVICES EMPLOYING CELL ROLLING

(75) Inventors: Jeffrey M. Karp, Brookline, MA (US); Michael J. Moore, New Orleans, LA (US); Ali Khademhosseini, Cambridge, MA (US); Robert S. Langer, Newton, MA (US); Seungpyo Hong, Naperville, IL (US)

Correspondence Address: CHOATE, HALL & STEWART LLP, TWO INTERNATIONAL PLACE, BOSTON, MA, 02110, US

(73) Assignee: MASSACHUSETTS INSTITUTE TO TECHNOLOGY, Cambridge, MA (US)

(21) Appl. No.: 12/596,405

(22) PCT Filed: Apr. 18, 2008
(86) PCT No.: PCT/US08/60934

§ 371 Date: Nov. 19, 2009

Related U.S. Application Data
(60) Provisional application No. 60/912,604, filed on Apr. 18, 2007.
(60) Provisional application No. 60/969,315, filed on Aug. 31, 2007.

(51) Int. Cl. A61K 9/00; C12N 5/00; A61K 35/12; A61P 9/00; A61P 35/00

(52) U.S. Cl. 424/422; 435/325; 435/402; 424/93.7

(57) ABSTRACT

In various aspects, the present invention provides surfaces and materials for cell rolling applications, methods of making such surfaces and materials, and devices having such surfaces and materials. In some embodiments, the present invention provides surfaces with at least partial coatings of an ordered layer of cell adhesion molecules, or fragments, analogs, or modifications thereof, covalently bound to the surface of the substrate through an immobilization moiety. In some embodiments, the layer of a cell adhesion molecules further comprises a cell modifying ligand that can be targeted, e.g., to one or more specific cell types.

### CROSS-REFERENCE TO RELATED APPLICATION

This application claims the benefit of, and priority to, U.S. Provisional Patent Application 60/912,604, filed Apr. 18, 2007, and to U.S. Provisional Patent Application 60/969,315, filed Aug. 31, 2007 which are hereby incorporated herein by reference.

### BACKGROUND

Cell rolling is an important physiological and pathological process that is used to recruit specific cells in the bloodstream to a target tissue. For example, cell rolling along vascular endothelium in viscous shear flow is of primary biological importance, given its role in recruitment of leukocytes to sites of inflammation, homing of hematopoietic progenitor cells after intravenous injection, tumor cell metastasis and other inflammatory processes.

Cell rolling is a receptor-ligand mediated event that initiates an adhesion process to a target tissue through a reduction in cell velocity followed by activation, firm adhesion, and transmigration. The rolling response is primarily mediated by a family of transmembrane domain-based glycoprotein receptors called selectins, which are expressed on the surfaces of leukocytes and activated endothelial cells. Selectins bind to carbohydrates via a lectin-like extracellular domain. The broad family of selectins is divided into L-selectin (CD62L), E-selectin (CD62E), and P-selectin (CD62P). L-selectin (74-100 kDa) is found on most leukocytes and can be rapidly shed from the cell surface. E-selectin (100 kD) is transiently expressed on vascular endothelial cells in response to IL-1 beta and TNF-alpha. P-selectin (140 kDa) is typically stored in secretory granules of platelets and endothelial cells.

For example, the adhesion mechanism that mediates leukocyte rolling on the vascular endothelium is often referred to as cell rolling. This mechanism involves the weak affinity between P-selectin and E-selectin (expressed on vascular endothelial cells) and selectin-binding carbohydrate ligands (expressed on circulating hematopoietic stem cells (HSC) and leukocytes). Once ‘captured’, cells roll slowly over the surface, in contrast to uncaptured cells, which flow rapidly in the bulk fluid.

### SUMMARY

Cell rolling is useful for uncovering fundamental biological information and, as described herein, for capturing and/or separating cells based on their cell rolling properties. Most cell rolling studies to date have employed random placement of selectins onto a 2-D substrate utilizing protein physisorption. The stability of physisorbed selectins is weak, as adsorbed proteins tend to rapidly desorb from the surfaces. This instability and lack of control over selectin distribution hampers practical application of cell rolling, e.g., for cell separation. In addition, physisorption does not afford a high degree of control over the presentation of selectins, which may hinder the ability to mimic relevant complexities of the in situ rolling response and to design efficient and effective separation tools.

In some embodiments, the methods described herein improve the exploitation of cell rolling processes for biomedical applications, e.g., those involving the capture and separation of specific cell types. As discussed herein, this is achieved in part by using covalent attachment methods to coat surfaces with cell adhesion molecules. These inventive covalent attachment methods have advantages, including longer functional stability and better control over the density and orientation of the cell adhesion molecules.

In some embodiments, functionalized surfaces for cell separation applications are provided. In some embodiments, cell separation can be achieved, (e.g., for clinical and research applications) without significantly affecting the cell surface antigen profile, and/or to facilitate cell isolation for stem cell and cancer cell therapies. In some embodiments, by using selectins that bind weakly as compared to antibodies, cells may roll over a surface without becoming permanently bound to it. In some embodiments, the present invention provides materials, surfaces, methods of making such materials and/or surfaces, and devices comprised of such surfaces and/or materials for controlling the movement of cells within the bloodstream. In some embodiments, devices for use with blood flow include those external to the body such as, e.g., AV shunts.

In some embodiments, implantable and/or injectable devices are provided which are comprised of materials and/or surfaces of the present invention that facilitate increasing the specificity of protein adsorption to the device. For example, there is a great demand for biocompatible materials that express specific ligands on their surfaces to regulate biological behavior. Nevertheless, surfaces of most implanted biomaterials quickly become covered with proteins or other blood components that adsorb non-specifically to such surfaces, which can reduce the effectiveness of such surfaces to direct biological processes. Although methods for reducing adsorption of proteins exist, traditional surface-bound ligands are not effective in influencing cell function for an extended period of time. In some embodiments, surfaces and/or materials of devices of the present inventions dynamically express new ligands. Such dynamic expression may facilitate maintaining efficiency and/or efficacy to affect biological processes. In some embodiments, the ligand disrupts or induces one or more processes chosen from cell quiescence, cell proliferation, cell migration, cell de-differentiation, cell spreading, cell attachment, and cell differentiation.

The rolling velocity of each cell type is a function of local shear rate, the distribution of receptors on cell membranes, and the total number of receptors present on the cell, which may differ from one cell type to another. In some embodiments, the present invention provides methods and surfaces targeted to specific cells types (e.g. cancer cells, stem cells, etc.). In some embodiments, methods are provided for generating surfaces that enable greater control over the presentation and stability of cell adhesion molecules, which may allow one to model and/or interrogate more complex phenomena. Covalent immobilization of proteins offers great potential for enhanced control over presentation and stability of biomolecules on surfaces. Covalent immobilization of selectins is advantageous over conventional physisorption. Covalent immobilization can facilitate optimization of cell-material interactions by allowing control over density of the surface coating, spatial patterning, active site orientation, stability and shelf life, and topology. Such control may be used to achieve specific rolling characteristics and/or may be facilitated by linkers. Although covalent immobilization procedures for peptides and enzymes have been extensively studied for decades, covalent immobilization of large molecular weight biomolecules such as selectins present significant challenges due to increased binding to non-specific sites and due to the requirement for mild processing conditions to prevent protein inactivation.

The foregoing and other aspects, embodiments, and features of the invention can be more fully understood from the following description and the accompanying drawings.

### BRIEF DESCRIPTION OF THE DRAWINGS

FIG. 1 schematically illustrates cell rolling controlled through cell adhesion molecules at a material interface according to certain embodiments of the present invention.

FIG. 2 schematically illustrates immobilized ligand incorporated on the surface of and within the bulk of a degradable matrix.

FIG. 3 schematically illustrates ligand-releasing degradable particles within a non-degradable or slowly degrading matrix.

FIG. 4 schematically illustrates ligand-containing particles that degrade slowly within a matrix that degrades more quickly.

FIG. 5 schematically illustrates endothelial cells stimulated to produce surface ligand locally through release of factors from an implantable (e.g., a stent) or injectable device.

FIG. 6 schematically illustrates a vascularized matrix within an ectopic site (e.g., peritoneal cavity) stimulating endothelial expression of ligand to direct cell function and/or to direct specific cell invasion (e.g., cancer, stem cells, etc.) into a matrix.

FIGS. 7A-C schematically illustrate surface preparation via various synthetic routes where, respectively, P-selectin was immobilized on amine (FIG. 7A), aldehyde (FIG. 7B), and epoxy (FIG. 7C) functionalized glass surfaces through a PEG linker (NH₂—PEG-COOH). On the amine glass, NH₂—PEG-COOH and P-selectin were pre-activated by EDC and NHS in solution before they were placed on the surfaces. For covalent immobilization on aldehyde and epoxy surfaces, carboxylated groups on the PEGylated surfaces were pre-activated using EDC/NHS and P-selectin was conjugated on top of the PEGylated glass surfaces. For comparison of surface stability with physical adsorption of P-selectin, plain glass substrate as well as PEGylated aldehyde and epoxy surfaces were employed without pre-activation with EDC/NHS.

FIG. 8 is a schematic diagram depicting preparation of microsphere conjugates and their rolling on a P-selectin-coated surface.

FIGS. 9A-D present data comparing measurements of surface stability detected through microsphere rolling where a solution of 1.0×10⁵ microspheres/ml was perfused at 0.24 dyn/cm² of shear stress. FIG. 9A presents data on aldehyde surface microsphere velocities. Velocities were normalized with respect to PEGylated physisorbed surface controls and are plotted as shown in FIG. 9B. FIG. 9C presents data on epoxy surface microsphere velocities. Normalized velocities are plotted as shown in FIG. 9D. For FIGS. 9A and 9B, data is presented on microsphere velocities on PEGylated aldehyde glass (▪) and on P-selectin immobilized on the PEGylated aldehyde surface without EDC/NHS pre-activation () and with EDC/NHS pre-activation (▴). For FIGS. 9C and 9D, data is presented on microsphere velocities on PEGylated epoxy glass (▪), on P-selectin adsorbed on plain glass (), and on P-selectin immobilized on the PEGylated epoxy surface without EDC/NHS pre-activation (▴) and with EDC/NHS pre-activation (▾). Although surfaces prepared on aldehyde glass do not show enhanced stability regardless of EDC/NHS pre-activation, P-selectin-immobilized surfaces prepared on the PEGylated epoxy glass pre-activated using EDC/NHS exhibit significantly enhanced stability. All the rolling dynamic data is represented as mean±SEM.

FIGS. 10A-C depict representative phase contrast micrographs of neutrophil rolling adhesion on P-selectin-adsorbed substrates. A still image of rolling adhesion of neutrophils on a P-selectin-adsorbed surface on plain glass is shown in FIG. 10A, and PEGylated epoxy glass slides without (FIG. 10B) or with (FIG. 10C) pre-activation using EDC/NHS are also depicted. 2.5×10⁵/ml of neutrophil solution was perfused on the 28-day-old P-selectin surface under 1 dyn/cm² of shear stress. A total magnification of 100× as applied and all scale bars indicate 100 μm.

FIGS. 11A-C present data on the rolling dynamics of neutrophils on P-selectin-immobilized surfaces under shear flow where 2.5×10⁵/ml of neutrophil solution was perfused on 3 or 28-day-old P-selectin surfaces under wall shear stresses from 1 to 10 dyn/cm². Rolling fluxes (FIG. 11A) and rolling velocities (FIG. 11C) of neutrophils were measured for each condition. FIG. 11B presents data on relative rolling fluxes on 28-day-old P-selectin surfaces at 3 dyn/cm². Mean values of fluxes from 3-day-old surfaces are each set to 100% and data from the 28 day-old surface are expressed as mean±SEM (%).

FIGS. 12A-B depict schematic diagrams of P-selectin immobilization on a) mixed SAMs (self-assembled monolayers) of OEG-COOH/OEG-OH at different ratios using the EDC/NHS chemistry and b) mixed SAMs of OEG-NH₂/OEG-OH using sulfo-SMCC ((sulfo-succinimidyl-4-[N-maleimidomethyl]cyclohexane-)-carboxylate) as a linker for protein orientation. P-selectins immobilized through amide bonds (depicted in FIG. 12A) and through thioether bonds (depicted in FIG. 12B) have, respectively, unoriented and oriented conformations on the surfaces. “OEG” refers to Oligo(Ethylene Glycol).

FIGS. 13A-B depict schematic diagrams of biotinylation of P-selectin using maleimide-PEG-biotin (FIG. 13A) and immobilization of the biotinylated P-selectin on a mixed SAM of OEG-biotin/OEG-OH through streptavidin (FIG. 13B). The maleimide group in maleimide-PEG-biotin reacts specifically with the single cysteine residue of P-selectin. This ensures that all P-selectin molecules are oriented in the same manner once the biotin group in maleimide-PEG-biotin interacts with the mixed SAM through streptavidin.

FIG. 14 presents SPR (surface plasmon resonance) sensorgram data comparing immobilization stability between covalently bound (with EDC/NHS activation) and physisorbed (without EDC/NHS activation) P-selectin on a mixed SAM of OEG-COOH:OEG-OH (3:7) using SPR. The following steps were performed: a) EDC/NHS activation, b) P-selectin immobilization, c) washing with PBS, and d) washing with Tris-HCl buffer. The amount of P-selectin immobilized was determined by subtracting the baseline (I) from the final wavelength shift (II).

FIGS. 15A-B present SPR sensorgram data of P-selectin immobilization with density controlled. By changing the ratio between OEG-COOH and OEG-OH, the amount of P-selectin immobilized is controlled (see data in FIG. 15A) and is proportional to the concentration of OEG-COOH (see data in FIG. 15B). Amount of immobilized P-selectin were measured from three independent channels at each condition. Error bars in FIG. 15B represent standard deviations.

FIG. 16 presents SPR sensorgram data for immobilization of P-selectin on a sulfo-SMCC)-coated chip surface. The SMCC-coated surface specifically allows P-selectin to be immobilized. Specificity was confirmed in an SPR experiment during which a >50 times excess of BSA was flowed. The wavelength shift for BSA binding (˜1 nm), was greatly lower than the shift observed for P-selectin (˜12 nm).

FIGS. 17A-B presents data on the effect of P-selectin orientation on antibody binding. FIG. 17A presents SPR sensorgrams of P-selectin immobilization on 3 different channels after flowing streptavidin into the channels to create specific binding sites for biotinylated P-selectin. FIG. 17B presents a comparison of antibody binding on unoriented P-selectin (using EDC/NHS chemistry) and oriented P-selectin (using thiol specific biotin-streptavidin chemistry) surfaces. Amounts of immobilized P-selectin were comparable for oriented and unoriented P-selectin, with both types of surfaces showing a wavelength shift of about 12 nm.

FIG. 18 schematically illustrates pre-activation of carboxylic ends of P-selectin using EDC, followed by either direct conjugation of the protein to the glass substrate or immobilization of the protein through a PEG linker.

FIG. 19 schematically illustrates a synthetic route for P-selectin-embedded PEG hydrogels.

FIG. 20 schematically illustrates a preparation of a dextran-based 3-D hydrogel matrix containing covalently immobilized P-selectin.

FIG. 21 schematically illustrates TRAIL conjugation to a glass substrate (2-D) and to a PEG hydrogel (3-D).

FIGS. 22A-B present data on the specific interaction between P-selectin and surface bound ligand (sLe^{x}) on microspheres of Example 8.

FIGS. 23A-D depict fluorescence microscopy images of P-selectin antibody-FITC conjugate of Example 8 incubated on untreated amine glass and amine glass substrates (FIG. 23A) with 5 μg (FIG. 23B); 10 μg (FIG. 23C), and 20 μg (FIG. 23D) of P-selectin.

### DEFINITIONS

The terms “about” and “approximately,” as used herein in reference to a number, generally includes numbers that fall within a range of 5%, 10%, or 20% in either direction of the number (greater than or less than the number) unless otherwise stated or otherwise evident from the context (except where such number would exceed 100% of a possible value).

The term “adsorb” is used herein consistently with its generally accepted meaning in the art, that is, to mean “to collect by adsorption.” “Adsorption” refers to the process by which specific gasses, liquids or substances in solution adhere to exposed surfaces of materials, usually solids, with which they are in contact.

The term “cell adhesion molecule,” as used herein, generally refers to proteins located on cell surfaces involved in binding (via cell adhesion) of the cell on which it is found with other cells or with the extracellular matrix. Examples of cell adhesion molecules include, but are not limited to, full-length, fragments of, analogs of, and/or modifications of selectins (e.g., E-selectins, P-selectins, L-selectins, etc.), integrins (e.g., ITGA4, etc.), cadherins (e.g., E-cadherins, N-cadherins, P-cadherins, etc.), immunoglobulin cell adhesion molecules, neural cell adhesion molecules, intracellular adhesion molecules, vascular cell adhesion molecules, platelet-endothelial cell adhesion molecules, L 1 cell adhesion molecules, and extracellular matrix cell adhesion molecules (e.g., vitronectins, fibronectins, laminins, etc.). As used herein, the term “cell adhesion molecule” also encompasses other compounds that can facilitate cell adhesion due to their adhesive properties. In some embodiments of the invention, aptamers, carbohydrates, peptides (e.g., RGD (arginine-glycine-aspartate) peptides, etc.), and/or folic acid, etc. can serve as cell adhesion molecules. As used herein, such compounds are encompassed by the term “cell adhesion molecule.” As used herein, terms referring to cell adhesion molecules including, but not limited to, “cell adhesion molecule,” “selectin,” “integrin,” “cadherin,” “immunoglobulin cell adhesion molecule,” “neural cell adhesion molecules,” “intracellular adhesion molecules,” “vascular cell adhesion molecules,” “platelet-endothelial cell adhesion molecules,” “L1 cell adhesion molecules,” “extracellular matrix cell adhesion molecules,” encompass full length versions of such proteins as well as functional fragments, analogs, and modifications thereof, unless otherwise stated. Likewise, terms referring to specific cell adhesion molecules including, but not limited to, “E-selectin,” “P-selectin,” “L-selectin,” “ITGA4,” “E-cadherin,” “N-cadherin,” “P-cadherin,” “vitronectin,” “fibronectin,” “laminin,” etc., also encompass full length versions of such proteins as well as functional fragments, analogs, and modifications thereof, unless otherwise stated. As used herein, the term “cell adhesion molecule” does not encompass antibodies.

The term “cell modifying ligand,” as used herein, generally refers to molecules that are capable of modifying the biological behavior of a cell. For example, a protein that triggers a molecular signal within a cell (e.g., expression of another protein) is a cell modifying ligand.

The term “oriented,” as used herein, is used to describe molecules (e.g., cell adhesion molecules, etc.) having a definite or specified spatial orientation, that is, a non-random orientation. For example, cell adhesion molecules are “oriented” on a surface if a substantial portion of the cell adhesion molecules on the surface have a particular spatial orientation with respect to the surface. In certain embodiments of the invention, the “substantial portion” comprises at least 50% of the molecules on the surface.

The term “unoriented,” as used herein, is used to describe molecules (e.g., cell adhesion molecules, etc.) having no particular or specified orientation, that is, a random orientation. For example, cell adhesion molecules may be described as “unoriented” on a surface if the cell adhesion molecules generally do not have a defined orientation with respect to the surface.

The term “ordered layer,” as used herein, refers to a layer having a property which is substantially uniform, periodic, and/or patternwise over at least 50% of the layer. In some embodiments, an ordered layer has one or more features chosen from a substantially uniform density and a substantially uniform spatial orientation of the cell adhesion molecules or fragments, analogs, or modifications thereof. In some embodiments, an ordered layer has one or more features chosen from a patternwise distribution, a patternwise density, and a patternwise spatial orientation of the cell adhesion molecules. In some embodiments, the ordered layer of cell adhesion molecules allows a velocity of cell rolling over the ordered layer that is substantially proportional to the shear stress applied to the ordered layer.

The term “physisorb” is used herein consistently with its generally accepted meaning in the art, that is, “to collect by physisorption.” “Physisorption” refers to adsorption that does not involve the formation of chemical bonds.

The term “self-assembled monolayer” (abbreviated as “SAM”), as used herein, refers to a surface consisting of a single layer of molecules on a substrate that can be prepared by adding a solution of the desired molecule onto the substrate surface and washing off the excess.

### DETAILED DESCRIPTION

In some embodiments, the present invention provides surfaces with at least partial coatings of an ordered layer of a cell adhesion molecule which is bound to the surface of the substrate through a covalent bond.

In some embodiments, the cell adhesion molecules are bound to the surface of the substrate through interactions that are entirely covalent. In some embodiments, the cell adhesion molecules are bound to the surface of the substrate through interactions that include one or more non-covalent bonds. For example, the inventive methods may employ a ligand/receptor type interaction to indirectly link a cell adhesion molecule to the surface of the substrate. Any ligand/receptor pair with a sufficient stability and specificity to operate in the context of the inventive methods may be employed. In the Examples, we describe methods in which streptavidin molecules were used to form non-covalent bridges between biotinylated selectins and a mixed SAM of OEG-biotin/OEG-OH that is covalently bonded to a substrate surface. The strong non-covalent bond between biotin and streptavidin allows for association of the selectin with the SAM and thus with the substrate surface. Other possible ligand/receptor pairs include antibody/antigen, FK506/FK506-binding protein (FKBP), rapamycin/FKBP, cyclophilin/cyclosporin, and glutathione/glutathione transferase pairs. Other ligand/receptor pairs are well known to those skilled in the art.

A variety of cell adhesion molecules can be used in the practice of certain embodiments of the present invention. In some embodiments, the layer of cell adhesion molecules comprises cell adhesion molecules having a dissociation constant (K_{D}) for interaction with one or more cell surface moieties (e.g., proteins, glycans, etc.) that is greater than about 1×10⁻⁸ mole/liter (M). In some embodiments, the layer of cell adhesion molecules comprises cell adhesion molecules having a dissociation constant (K_{D}) for interaction with one or more cell surface moieites that is in the range of about 1×10⁻⁴ molar to about 1×10⁻⁷ M, inclusive. It will be appreciated that the behavior of cells on the coated surface will depend in part on the dissociation constant. In some embodiments, a coated surface can be used to capture cells. In some embodiments, e.g., by controlling the density and/or patterning of immobilized cell adhesion molecules, a coated surface can be used to reduce the velocity of moving cells that interact with the substrate rather than, e.g., promoting them to stop and adhere. In some embodiments, the substrate further comprises molecules that may facilitate stopping cells that roll over the ordered layer. Molecules that have strong interactions with cell surface ligands, such as antibodies, may be useful in such embodiments.

In general, any cell adhesion molecule may be used. Examples of cell adhesion molecules useful in certain embodiments of the present invention include, but are not limited to, full-length, fragments of, analogs of, and/or modifications of selectins (e.g., E-selectins, P-selectins, L-selectins, etc.), integrins (e.g., ITGA4, etc.), cadherins (e.g., E-cadherins, N-cadherins, P-cadherins, etc.), immunoglobulin cell adhesion molecules, neural cell adhesion molecules, intracellular adhesion molecules, vascular cell adhesion molecules, platelet-endothelial cell adhesion molecules, L1 cell adhesion molecules, and extracellular matrix cell adhesion molecules (e.g., vitronectins, fibronectins, laminins, etc.). In some embodiments, aptamers, carbohydrates, peptides (e.g., an RGD peptide), folic acid, etc. can serve as cell adhesion molecules.

Any covalent chemistry may be used to covalently attach cell adhesion molecules to a substrate surface. Those skilled in the art will appreciate that the methods described in the Examples are exemplary and could be readily modified based on knowledge in the art. In some embodiments, cell adhesion molecules are attached to a surface through one or more linker moieties. In some embodiments, a linker moiety is bound to the cell adhesion molecule at one of its ends and to the surface of the substrate at another end. In general, the bond between the linker moiety and the surface is covalent. The bond between the linker moiety and the cell adhesion molecule may be covalent or non-covalent (e.g., if it involves a ligand/receptor pair as discussed above). Without limitation, in some embodiments, the linker moiety comprises one or more of a dextran, a dendrimer, polyethylene glycol, poly(L-lysine), poly(L-glutamic acid), poly(D-lysine), poly(D-glutamic acid), polyvinyl alcohol, and polyethylenimine. In some embodiments, the linker moiety comprises one or more of an amine, an aldehyde, an epoxy group, a vinyl, a thiol, a carboxylate, and a hydroxyl group. In some embodiments, the linker moiety includes a member of a ligand/receptor pair and the cell surface molecule has been chemically modified to include the other member of the pair.

In addition to improving the long term stability and behavior of the coated surface, the use of covalent bonding instead of physisorption, enables one to control the density, pattern and orientation of cell adhesion molecules on the substrate surface. For example, the density will depend on the density of groups on the surface which are available for covalent bonding. Similarly, the pattern will depend on the pattern of groups on the surface which are available for covalent bonding. Methods are well known in the art for preparing surfaces with different densities and patterns of suitable groups for covalent bonding (e.g., see Rusmini et al. Protein immobilization strategies for protein biochips. Biomacromolecules 2007 June; 8(6):1775-89. and Leckband et al. An approach for the stable immobilization of proteins. Biotechnology and Bioengineering 1991; 37(3):227-237, the entire contents of both of which are incorporated herein by reference). In some embodiments, the density of cell adhesion molecules ranges from about 10 ng/cm² to about 600 ng/cm². In some embodiments, the density of cell adhesion molecules is greater than about 30 ng/cm². For example, in some embodiments, the density of cell adhesion molecules ranges from about 30 ng/cm² to about 360 ng/cm². In some embodiments, the density of cell adhesion molecules ranges from about 50 ng/cm² to about 300 ng/cm². In some embodiments, the density of cell adhesion molecules ranges from about 100 ng/cm² to about 200 ng/cm².

In some embodiments, the orientation of cell adhesion molecules on the surface can also be controlled. This can be advantageous, e.g., because the cell adhesion molecule only interacts with cells if a particular region is accessible to the cells. For example, as discussed in the Examples, P-selectin includes a single cysteine residue. As a result, if P-selectin is attached to the surface via a linker moiety that reacts specifically with cysteine, all P-selection molecules will be attached to the surface with the same orientation. In general, this approach can be applied whenever the cell adhesion molecule includes a unique group. In some embodiments, a cell adhesion molecule can be engineered or chemically modified using methods known in the art to include such a unique group (e.g., a particular amino acid residue) at a position that provides an optimal orientation. For example, a suitable amino acid residue can be added at the C- or N-terminus of protein based cell adhesion molecules.

In some embodiments, the cell adhesion molecules are synthesized and/or purified such that only a limited subset of the residues is able to react with reactive groups on the surface or on the linker. In some embodiments, there is only one group or residue on each cell adhesion molecule that can react with reactive groups on the surface or on the linker. For example, in some embodiments, cell adhesion molecules are synthesized and/or purified with protecting groups that prevent the residues to which they are attached from reacting with reactive groups on the surface or linker. In such embodiments, one or more residues in the cell adhesion molecule are not protected. Because the cell adhesion molecule can only attach to the surface or linker via the one or more unprotected residues, the cell adhesion molecule may attach to the surface or linker in a specific orientiation. In some embodiments, the protective groups are removed after attachment of the cell adhesion molecule to the surface or linker. (See, e.g., Gregorius et al. Analytical Biochemistry 2001 Dec. 1; 299(1):84-91, the entire contents of which are incorporated herein by reference.)

Depending on the intended use of the coated surface, the layer of cell adhesion molecules may include a single cell adhesion molecule or a combination of different cell adhesion molecules. In some embodiments, cell modifying ligands may be co-immobilized with cell adhesion molecules. In general, a cell modifying ligand may be attached to the surface in a similar fashion to the cell adhesion molecule (e.g., using the same linker moiety). In certain embodiments, the cell modifying ligand may be attached using a different covalent attachment method. In certain embodiments, the cell modifying ligand may be attached non-covalently. In certain embodiments, the ordered layer comprises at least one cell modifying ligand that is covalently attached to the surface and least one cell modifying ligand that is non-covalently attached to the surface. For example, to induce apoptosis or programmed cell death, tumor necrosis factor (TNF)-related receptor apoptosis-inducing ligand (TRAIL) may be co-immobilized with a cell adhesion molecule. TRAIL specifically binds to TNF receptors 5 and 6 and is expressed on cancer cells but not normal cells. Cell modifying ligands such as TRAIL and/or other chemotherapeutic agents can be co-immobilized with a cell adhesion molecule to impart signals to kill or arrest growth of cancer cells. It will be appreciated by those skilled in the art that other cell modifying ligands can be immobilized and/or presented on and/or within the substrate to influence the behavior of cells that interact with the cell adhesion molecules. For example, fibroblast growth factor 2 (FGF-2) can be presented to facilitate maintaining cells in an undifferentiated state. As a further example, bone morphogenic protein 2 (BMP-2) can be presented to stimulate osteogenic differentiation of stem cells, etc. Combinations of cell modifying ligands can also be used together.

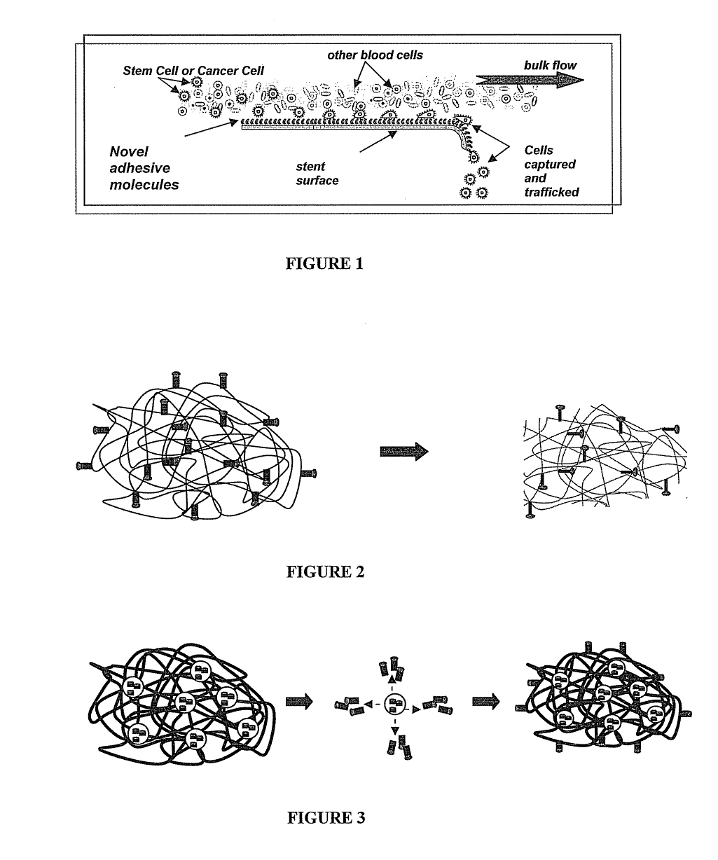

In some embodiments, the present invention provides coated surfaces that influence rolling behavior of cells. For example, FIG. 1 schematically illustrates cell rolling controlled via cell adhesion molecules on a surface according to various embodiments of the present invention. Such coated surfaces have a wide range of applications including, but not limited to, therapeutic applications. For example, these coated surfaces can be used, e.g., to deliver and/or expose a cell modifying ligand to specific cell types and/or to capture cells for future use (e.g., cancer cells, stem cells, etc.). They can also be used for the disposal of specific cells (e.g., cancer cells), etc. As a further example, these coated surfaces and devices comprising them can be used to separate cells into subpopulations. Subpopulations of cells may then be quantified and/or collected for further uses.

In some embodiments, of the coated surfaces are present on substantially degradable substrates that are bulk modified with cell modifying ligands. The degradable substrates can be made, e.g., from hydrogels and/or hydrophobic materials such as polymers (see, e.g., FIG. 2). In some embodiments, surface erodible polymers such as poly(glycerol sebacic acid), polyanhydrides, poly(diol citrates), or combinations thereof are used. These degradable substrates may also be combined with other hydrogel materials (e.g., poly(ethylene glycol), hyaluronic acid, etc.) to create more hydrophilic materials. In some embodiments, new cell modifying ligands are exposed as these substrates erode.

In some embodiments, certain coatings of the present invention are applied to substantially non-degradable substrates (or slowly degrading substrates) that have entrapped cell modifying ligands in the bulk either alone or within releasing vehicles (e.g., nanoparticles, microparticles, combinations thereof, etc.). In some embodiments, a released ligand is transported to the surface of the substrate and adsorbed, thus replenishing the surface with active ligand (see, e.g., FIG. 3).

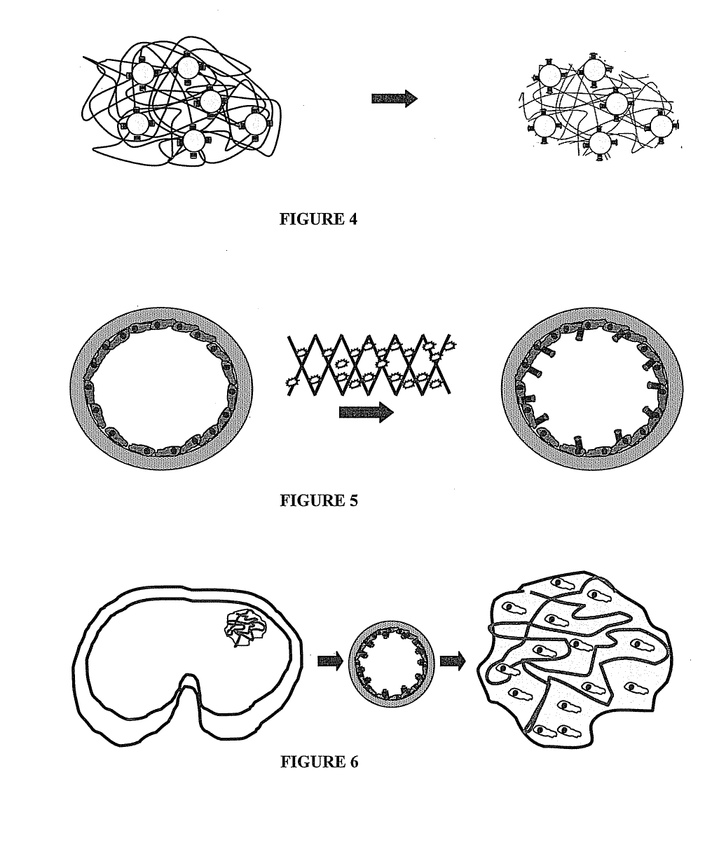

In some embodiments, certain coatings of the present invention are applied to substantially degradable substrates containing particles or regions of more slowly degrading materials containing entrapped and/or surface-bound ligand. As the bulk of the substrate degrades, particles containing ligand are exposed that serve to create a patterned surface of ligand (see, e.g., FIG. 4).

In some embodiments, certain coatings of the present invention are applied to an implantable and/or injectable substrate. For example, in various embodiments, cells lining blood vessel walls (e.g., endothelial cells, etc.) are stimulated to produce cell modifying ligands on their surfaces that aid in controlling cell function locally via the implanted or injected material (see, e.g., FIG. 5).

In some embodiments, certain coated substrates of the present invention are implanted into an ectopic site to serve as a niche environment for stimulating vascularization. After such an environment has been vascularized, ligands released within the substrate stimulate cells lining vessels within the material to produce ligands on their surface that can modulate cell function (see, e.g., FIG. 6). For example, in some embodiments of the invention, by slowing cell movement over the coated substrate with one or more cell adhesion molecules, ligands can be directed to these cells to influence cell behavior (e.g., slow cell growth, destroy cells such as cancer cells, direct cell fate, direct cell differentiation, induce cell de-differentiation, etc.). In some embodiments, cells can also invade the substrate and become entrapped. This route could be useful, e.g., for achieving a high surface area of contact between rolling cells and the endothelial surface. Cells that are entrapped may be, for example circulating cells such as metastasizing cancer cells, stem cells, progenitor cells (such as, e.g., endothelial progenitor cells), and combinations thereof. For cancer applications, this can in some embodiments facilitate targeting metastasizing cells to form a tumor in a particular region of the body that can be easily removed. In some embodiments, the implanted material can be used to capture circulating stem cells, e.g., to facilitate harvesting them.

In certain embodiments, the cell adhesion molecule is a selectin expressed by endothelial cells that participate in localization and/or extravasation of cancer cells. Such selectin expression may help target metastasizing cancer cells to particular organs. (For a review, see, e.g., Gout S. et al. Selectins and selectin ligands in extravasation of cancer cells and organ selectivity of metastasis. Clinical and Experimental Metastasis 2007, the entire contents of which are incorporated herein by reference.) For example, in some embodiments, the cell adhesion molecule is a selectin expressed on the surface of blood vessels within the bone marrow that may be responsible for localization of metastatic cancer cells (such as, e.g., prostate cancer cells).

In certain embodiments, prefabricated vascularized matrices are created that are designed to influence cell rolling behavior, and such vascularized matrices may be implanted (for example, in a patient) to achieve one or more of the outcomes described herein. Such matrices can be created with the patient's own endothelial cells that can be harvested from specific organs such as bone marrow.

In some embodiments, surfaces, materials and devices of the present invention can facilitate development of research, diagnostic and/or therapeutic products for, among other things, metastatic cancer and/or for stem cell therapy. Examples of such potential applications include, but are not limited to, isolation modules to collect cells from blood samples for in vitro study, implants in the vasculature that deliver apoptotic signals to cancer cells before they engraft at a distant site (i.e., metastasize), etc.

### EXAMPLES

### Example 1

### Comparison of Three Conjugation Chemistries

In the present Example, three conjugation chemistries were investigated. Amine, aldehyde, and epoxy functionalized glass substrates were tested using a parallel plate flow chamber to mimic physiologic flow conditions. The prepared surfaces were characterized by x-ray photoelectron scattering (XPS) and contact angle measurements. To prescreen each chemistry before conducting cell-based studies, we used 10 μm microspheres conjugated with Sialyl Lewis(x). It was found that among the three chemistries investigated, epoxy chemistry (which is stable at neutral pH in aqueous environments) achieved the longest term storage and the most bond stability without protein aggregation. The epoxy chemistry of the present Example led to significant enhancement in the stability of microsphere rolling. These results were validated through in vitro cell rolling experiments conducted in a manner substantially similar to protocols previously described. (See, e.g., King, M. R., “Scale invariance in selectin-mediated leukocyte rolling” in Fractals—Complex Geometry Patterns and Scaling in Nature and Society 12, 235-241 (2004), and King, M. R., Sumagin, R., Green, C. E., and Simon, S. I., “Rolling dynamics of a neutrophil with redistributed L-selectin” in Mathematical Biosciences 194, 71-79 (2005), the entire contents of both of which are herein incorporated by reference in their entirety).

### Materials

Recombinant Human P-selectin/Fc chimera (P-selectin) and mouse monoclonal antibody specific for human P-selectin (clone AK-4) were purchased from R&D systems (Minneapolis, Minn.). All the functionalized glass surfaces (plain, amine, aldehyde, and epoxy glass) were provided by TeleChem International, Inc (Sunnyvale, Calif.). Heterobifunctional poly(ethylene glycol) (NH₂—PEG-COOH) was acquired from Nektar (San Carlos, Calif.). All other chemicals were obtained from Sigma-Aldrich (St. Louis, Mo.). All the materials employed in this Example were used without further purification unless specified.

### Preparation of Surfaces

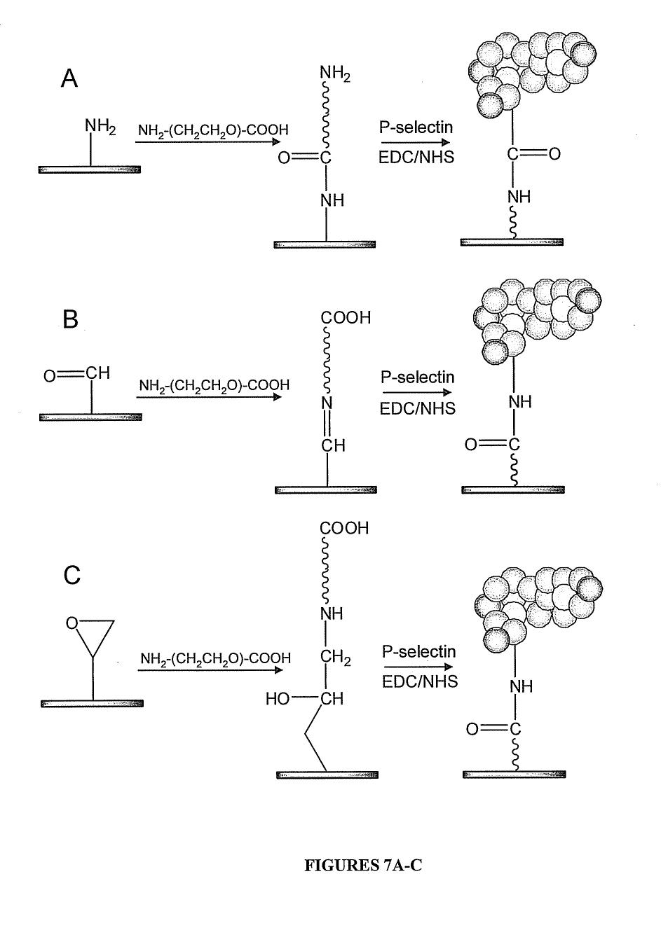

A synthetic route for surface preparation is illustrated in FIGS. 7A-C. Briefly, P-selectin immobilization was performed on four different glass substrates. Glass surface with physically adsorbed P-selectin was prepared on the plain glass. The plain glass substrate (SuperClean2®) was washed with PBS three times, 5 min for each was. 600 μL of P-selectin at a 5 μg/mL concentration was placed on top of the glass and incubated on a plate shaker for 18 hrs. For covalent immobilization of P-selectin, amine (SuperAmine-2®), aldehyde (SuperAldehyde2®), and epoxy (SuperEpoxy-2®) functionalized glass surfaces were employed. AFM (atomic force microscopy) analysis and other characterization results of all underlying glass substrates can be found at the arrayit.com website.

To ensure effective surface modification, all reagents were used in excess quantities. According to the supplier, SuperAmine-2® glass surfaces have 2×10¹³ reactive groups per mm² whereas SuperAldehyde2® and SuperEpoxy-2® glass surfaces have 5×10¹² reactive groups per mm². Therefore a total surface area of 10 cm² has approximately 2×10¹⁶ or approximately 5×10¹⁵ reactive groups. Reagents including NH₂—PEG-COOH were used with an excess molarity of 10-100× as described below.

For amine-functionalized glass surfaces, NH₂—PEG-COOH (500 μL at a concentration of 5 mg/mL) was pre-activated by adding 500 μL of a 1:1 mixture of 50 mM (1.9 mg/mL) 1-Ethyl-3-[3-dimethylaminopropyl]carbodiimide (EDC) and 50 mM (2.2 mg/mL) N-hydroxysuccinimide (NHS) in distilled water and incubating for 5 minutes, immediately followed by incubation on the glass surface at room temperature for 1 hour. One milliliter of P-selectin (5 μg/mL) was also pre-activated by EDC (19 μg) and NHS (22 μg) for 5 min, added on top of the PEGylated glass, and incubated at room temperature overnight. The glass surfaces were washed thoroughly with PBS at each step.

For aldehyde-functionalized glass surfaces, 600 μL of NH₂—PEG-COOH (5 mg/mL) were added onto the glass surface and incubated for 2 hours. After washing with PBS three times, some of the surfaces were treated by a 10× molar excess of sodium cyanoborohydride (5×10⁻⁶ mol) compared to the concentration of NH₂—PEG-COOH to reduce the unstable Schiff bases to stable secondary amines. EDC (160 μg) and NHS (180 μg) were added to 500 μL of PBS, and the EDC/NHS/PBS solution was incubated for 30 minutes on top of the surface to activate COOH groups. The EDC/NHS/PBS solution was removed from the surface and 600 μL of P-selectin (5 μg/mL) was immediately added and permitted to react at room temperature for 18 hours.

For epoxy-functionalized glass surfaces, NH₂—PEG-COOH was immobilized, activated by EDC/NHS, and reacted with P-selectin under the same conditions as for aldehyde-functionalized glass surfaces, except for the reduction reaction. (For epoxy-functionalized glass surfaces, stabilization by a reducing agent was not used).

For stability tests, surfaces were immersed in PBS and placed on a plate shaker at room temperature. Aged surfaces were compared to freshly prepared surfaces in subsequent flow chamber experiments.

### X-Ray Photoelectron Spectroscopy (XPS) and Contact Angle Measurement

Surfaces at each step were characterized by XPS and contact angle measurement (Table 1). XPS measurements were performed using an Axis Ultra X-ray Photoelectron spectrometer (Kratos Analytical, Manchester, UK) equipped with a monochromatic Al K-alpha source (1486.6 eV, 150 W) and a Hemispherical analyzer. The mass concentration % was obtained at a take-off angle of 20° at 80 eV pass energy and 0.2 eV step size.

Contact angles of double distilled water on surfaces were measured using a VCA2000 system (AST Products, Inc., Billerica, Mass.). Drops of 3 μL were deposited ontosample surfaces using a microsyringe attached to the system, and data were analyzed using VCA Optima XE software.

TABLE 1
Relative surface composition and contact angles of various surfaces

| | Plain Glass Substrate | | Aldehyde Glass Substrate | | | Epoxy Glass Substrate | | |
|---|---|---|---|---|---|---|---|---|
| | Plain | P-selectin | Aldehyde | PEGylated | P-selectin | Epoxy | PEGylated | P-selectin |
| C^{a} | 11% | 58% | 20% | 23% | 55% | 18% | 19% | 56% |
| N^{a} | 1% | 12% | — | 0% | 9% | 1% | 0% | 8% |
| O^{a} | 47% | 23% | 43% | 43% | 34% | 43% | 44% | 33% |
| Si^{a} | 37% | 7% | 30% | 33% | 2% | 32% | 37% | 3% |
| Contact | 34 ± 2 | 68 ± 4 | 49 ± 5 | 45 ± 5 | 66 ± 4 | 43 ± 6 | 50 ± 5 | 65 ± 5 |
| Angle (°)^{b} | | | | | | | | |

^{a}All standard deviations for XPS data (mass concentration %) were less than ±5% (pass energy 80 eV, step size 0.2 eV) at take-off angle 20° measured by XPS.
^{b}Contact angles of each surface were measured 4 times using double distilled water and expressed as mean ± SD.

### Preparation and Characterization of Microsphere Conjugates

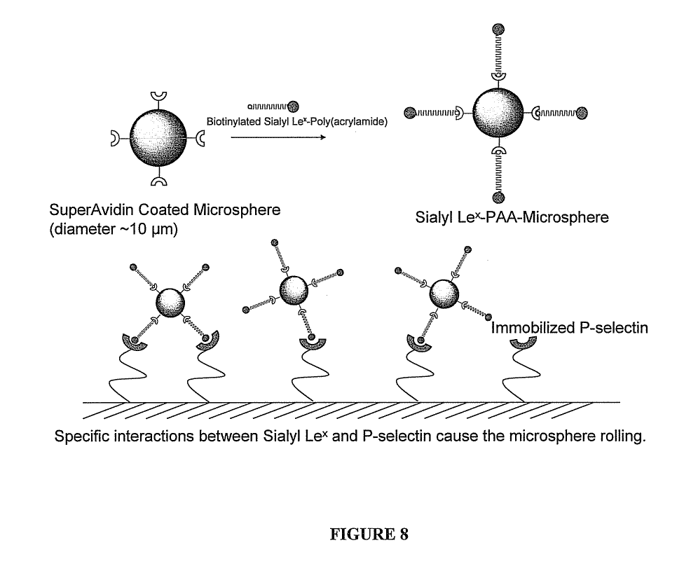

SuperAvidin™-coated microspheres with a diameter of 9.95 μm (Bangs Laboratories, Fishers, Ind.) were conjugated with multivalent biotinylated Sialyl Lewis(x)-poly(acrylamide) (sLe^{x}-PAA-biotin, Glycotech, Gaithersburg, Md.) to be used as a cell mimic for our pre-screening tests according to protocols described previously (FIG. 8). (See, e.g., King, M. R., and Hammer, D. A. “Multiparticle adhesive dynamics: Hydrodynamic recruitment of rolling leukocytes” in Proceedings of the National Academy of Sciences of the United States of America 98, 14919-14924 (2001), the entire contents of which are herein incorporated by reference in their entirety). Briefly, a 104.8 μl bead solution (containing 2×10⁶ beads) was dissolved into 1 ml of PBS containing 1% BSA (BPBS). The mixture was washed with BPBS three times by centrifugation at 10,000 rpm for 2 minutes. Four microliters of 1 mg/ml sLe^{x}-PAA-biotin (4 μg sLe^{x}) was added into the mixture and incubated for 1 hour at room temperature with occasional vortexing. The resulting solution was then washed again with BPBS three times by centrifugation at 10,000 rpm for 2 minutes. The final solution was resuspended in BPBS and diluted at a concentration of 1×10⁵ beads/ml to be used in adhesion experiments. In control experiments, native SuperAvidin™-coated microspheres (without sLe^{x} modification) at the same concentrations were also used to assess the velocity of non-interacting microspheres.

Flow Chamber Assay with Microsphere-Ligand Conjugates

A rectangular parallel-plate flow chamber (Glycotech) with a gasket of thickness of 250 μm and length 6 cm was placed on the glass surfaces with P-selectin. Flow rate-shear stress relationship was calculated based on the following equation 1.

(τₛ*)ₘₐₓ=(6×2.95μQ_{2-D}*)/H² (1)

Where, (τₛ*)ₘₐₓ is the maximum shear stress on the surfaces, μ is the viscosity of the fluid (water=0.01 dyn s/cm²), Q_{2-D}* is the flow rate per unit width in the system, and H is the height of the channel. All the flow chamber experiments using the microspheres were performed at a flow rate of 50 μL/min which is translated into a wall shear stress of 0.24 dyn/cm² in this system. Note that different conditions were used for cell-based experimentation.

For the microsphere experiment, 5×10⁵ ml⁻¹ of multivalent sLeₓ-coated microspheres were prepared in PBS containing 1% BSA and perfused into a flow chamber at a shear stress of 0.24 dyn/cm² using a syringe pump (New Era Pump Systems, Inc., Farmingdale, N.Y.). During each microsphere experiment, flow was interrupted for 1 minute, followed by image recording for 2 minutes. The flow was stopped to promote microsphere-surface contact via sedimentation. Images were taken every 5 seconds on an Axiovert 200 Zeiss microscope (Carl Zeiss, Thornwood, N.Y.) equipped with a camera controlled by a Hamamatsu camera controller (Hamamatsu, Japan) and velocities were calculated by measuring the displacement of each microsphere in subsequent images using AxioVision software version 3.1 (Carl Zeiss, Thornwood, N.Y.). Average velocities were obtained by averaging the velocities of at least 20 microspheres. All flow chamber experiments using microspheres were performed at a flow rate of 50 μL/min, which translates to a wall shear stress of 0.24 dyn/cm² in this system. Note that different conditions were used for cell-based experimentation. Rolling dynamic data is represented as mean±SEM.

Flow Chamber Assay with Neutrophils

Human blood was collected into a sterile tube containing sodium heparin (BD Biosciences, San Jose, Calif.) via venipuncture after obtaining informed consent. Neutrophils were then isolated by centrifugation (480×g at 23° C. for 50 min) with 1-Step Polymorphs (Accurate Chemical & Scientific Co., Westbury, N.Y.). After isolation, neutrophils were kept in sterile Hank's Balanced Salt Solution (pH 7.4) containing 0.5% human serum albumin, 2 mM Ca²⁺, and 10 mM HEPES until they were used in flow experiments. A rectangular parallel-plate flow chamber (Glycotech) with a gasket of thickness 127 μm and length 6 cm was placed on a P-selectin-immobilized glass surface. The assembled flow chamber was placed on an inverted microscope, Olympus IX81 (Olympus America Inc., Center Valley, Pa.) and the neutrophil solution, at a concentration of 2.5×10⁵/ml, was perfused into the chamber at different flow rates using a syringe pump (New Era Pump Systems, Inc.). The perfusion pump generated a laminar flow inside the flow chamber, allowing regulation of calculated wall shear stresses from 1 to 10 dyn/cm².

### Data Acquisition and Cell Tracking

A microscope-linked CCD camera (Hitachi, Japan) was used for monitoring neutrophil rolling interactions with adhesive P-selectin substrates. Rolling of neutrophils was observed using phase contrast microscopy and recorded on high quality DVD+RW discs for cell tracking analyses. Cell rolling videos were re-digitized to 640×480 pixels at 29.97 fps (frames per second) with ffmpegX software. Rolling fluxes and velocities of neutrophils interacting with immobilized P-selectin were then acquired using a computer-tracking program coded in ImageJ 1.37 m (NIH) and MATLAB 7.3.0.267 (R2006b) (Mathworks). A cell was classified as rolling if it rolled for more than 10 seconds while remaining in the field of view (864×648 μm² using a 10× objective (NA=0.30; Type: Plan Fluorite; Olympus America Inc.)) and if it translated at an average velocity less than 50% of the calculated free stream velocity of a non-interacting cell. This criteria was specific to the cell-based study. The free stream velocity was calculated using the theory of Goldman et al. (see, e.g., Gordon, M. Y., Marley, S. B., Davidson, R. J., Grand, F. H., Lewis, J. L., Nguyen, D. X., Lloyd, S., and Goldman, J. M., “Contact-mediated inhibition of human haematopoietic progenitor cell proliferation may be conferred by stem cell antigen, CD34.” in Hematol J 1, 77-86 (2000), the entire contents of which are herein incorporated by reference in their entirety). Rolling dynamic data was represented as mean±SEM of duplicate observations. Each observation was measured for 1 minute under each shear stress tested. To determine statistical significance among the data, p-values were calculated using a paired Student's t-test method.

### Results and Discussion

An early example of cell rolling studies was performed by Tim Springer's laboratory in 1991 using selectins within lipid bilayers (see, e.g., Lawrence, M. B., and Springer, T. A., “Leukocytes roll on a selectin at physiologic flow rates: distinction from and prerequisite for adhesion through integrins” in Cell 65, 859-73 (1991), the entire contents of which are herein incorporated by reference in their entirety). This model system was used to reproduce early leukocyte interactions with vascular endothelium. As discussed previously, since physisorbed proteins adhere mainly through weak intermolecular forces (e.g., van der Waals interactions) with an established equilibrium between adsorbed and free protein, these surfaces have limited stability and are only suitable for immediate or short term use. In the present Example, covalent immobilization of selectins using a variety of substrate chemistries to enhance stability and offer potential control of spatial orientation was explored.

### Amine Substrate Chemistry

As illustrated in FIGS. 7A-C, surfaces for covalent immobilization of P-selectin were pre-coated with NH₂—PEG-COOH. Heterobifunctional PEG was used to provide reactive sites for P-selectin and to produce non-fouling surfaces. P-selectin was covalently conjugated to PEGylated surfaces via amide bonds between carboxylate groups and primary amine groups.

Amine coupling is commonplace due to the availability of primary amines and carboxylates on surfaces of proteins. Amine reactive groups on a solid substrate (such as, for example, silanized glass) form covalent bonds with the carboxyl groups of PEG linkers, and amine groups of such linkers react with carboxyl termini of proteins. This chemistry was initially thought to be useful for enhanced orientation of P-selectin since the active site of the protein is known to be near the amine termini (the opposite end of the carboxyl termini). However, given the relatively low reactivity of amine groups, carboxyl termini on P-selectin must be activated by EDC and NHS for the reaction to occur. We found that EDC/NHS activation of P-selectin in solution led to aggregation of P-selectin, which resulted in undesirable formation of micron-sized particles. Since we were unable to curb substantially this aggregation by changing reaction conditions, we rationalized that this strategy was not suitable for enhancing control over P-selectin presentation.

We next investigated aldehyde chemistry, given that this chemistry does not require activation of P-selectin or PEG linkers in solution, as aldehyde groups on silanized glass possess a high reactivity towards amine groups. Aldehydes bind through Schiff base aldehyde-amine chemistry to amines on PEG. After activation of PEG with EDC/NHS, carboxylate termini on PEG react with amine groups within lysine residues of proteins or with the primary amine terminus.

As an additional strategy, P-selectin was immobilized on PEGylated epoxy-coated glass substrates. Such substrates have been widely used for protein conjugation, particularly in microarrays. Epoxy-coated slides are derivatized with epoxysilane, and proteins are covalently attached through an epoxide ring-opening reaction primarily with surface amino groups on proteins. In contrast to amine-based chemistry, it was found that with epoxy-based and aldehyde-based chemistries, EDC/NHS activation can be performed on the surfaces, which obviates substantial protein aggregation due to intramolecular loop formation and/or intermolecular interactions. In addition, epoxy-based chemistry has an added advantage over aldehyde chemistryn in that the reaction between epoxy and amine results in very stable bond formation. A stable bond can be also formed using aldehyde-based chemistry if the bond is reduced by a reducing agent such as sodium cyanoborohydride. However, this requires an additional step, and in our experiments, it reduced functionality of immobilized P-selectin.

Surface modifications were confirmed by XPS and contact angle measurements as shown in Table 1. Aldehyde and epoxy functionality was evidenced by increased carbon:oxygen ratios as compared to that of plain glass substrate. P-selectin immobilization (both physisorbed and chemically bound) was evident by an increase in nitrogen composition, decreased visibility of silicon in the underlying glass substrate, and increased contact angle. All surfaces treated with P-selectin had a high degree of coverage, as evidenced by the lack of visible underlying silicon. Furthermore, microspheres and cells encountered similar substrate properties, given the consistency in elemental composition and surface energy (contact angle values). Higher relative oxygen concentrations were observed on chemically immobilized P-selectin surfaces due, it is believed, to the presence of underlying PEG that are lacking on physisorbed substrates.

### Aldehyde-Based Chemistry

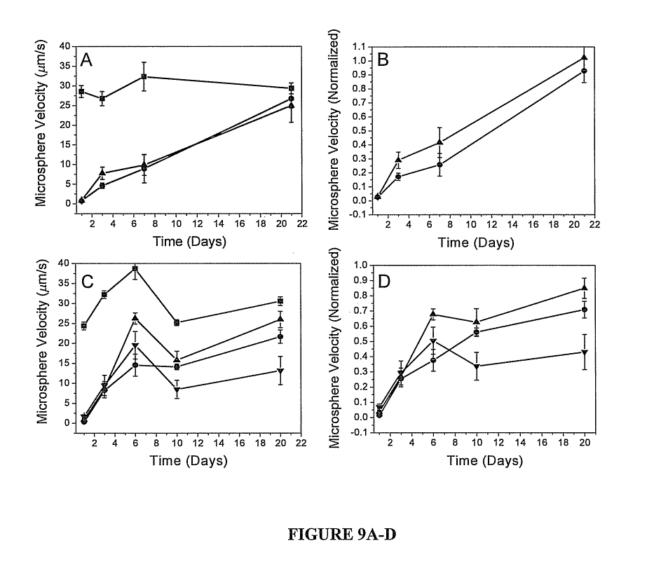

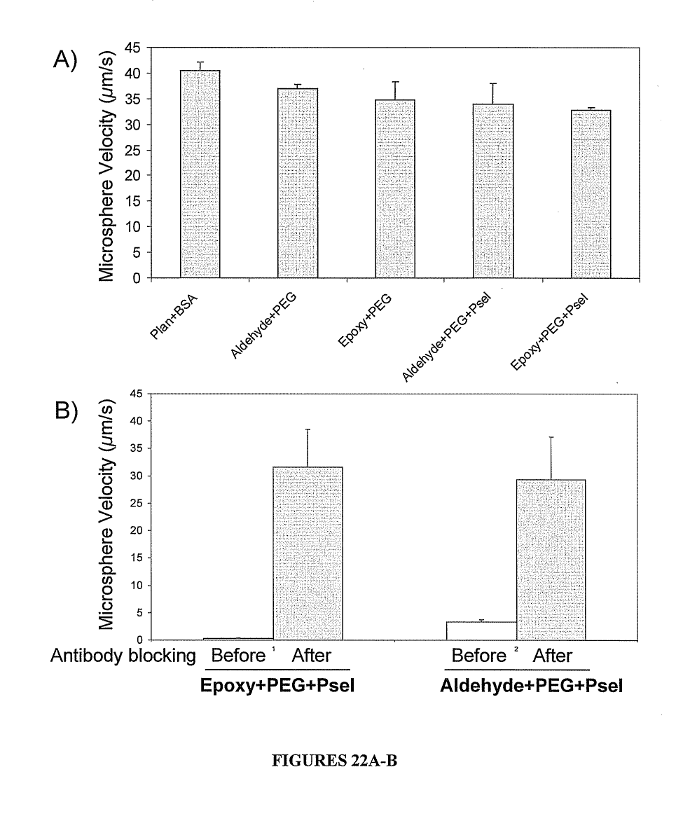

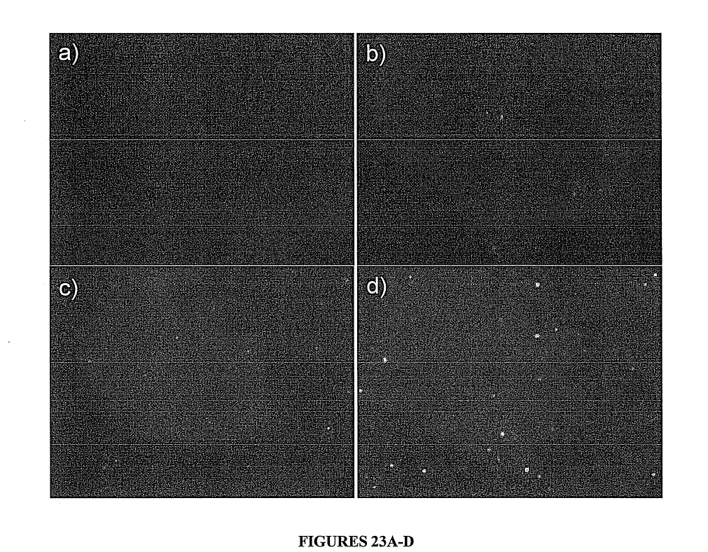

To test adhesion properties of prepared surfaces, microspheres conjugated with the ligand sLe^{x}-PAA-biotin were employed prior to testing with human cells. Adhesion properties of P-selectin immobilized surfaces were tested with the sLe^{x} microspheres using a flow chamber as previously described (see, e.g., King, M. R., and Hammer, D. A. “Multiparticle adhesive dynamics: Hydrodynamic recruitment of rolling leukocytes” in Proceedings of the National Academy of Sciences of the United States of America 98, 14919-14924 (2001), the entire contents of which are herein incorporated by reference in their entirety). As a control experiment, microspheres without ligands (sLe^{x}) demonstrated no rolling behavior (average velocities of 32-40 μm/s on the P-selectin coated surfaces) and surfaces without P-selectin did not reduce velocities of flowing microsphere conjugates (see, e.g., Example 8 and FIG. 22A). In addition, to block specific interaction between P-selectin and sLe^{x} on microspheres, P-selectin coated surfaces were post-treated using P-selectin antibody, followed by perfusion of microsphere conjugates into the flow chamber. After antibody treatment, the microsphere average velocities on P-selectin-coated surfaces increased from 0.4 to 31.6 μm/s and 3.4 to 29.2 μm/s on P-selectin immobilized epoxy and aldehyde surfaces, respectively (see, e.g., Example 8 and FIG. 22B). These results indicate that the observed velocity reduction on P-selectin-coated surfaces is solely due to a P-selectin-mediated interaction. Also, surfaces without P-selectin did not reduce velocities of flowing microsphere conjugates. All of the freshly made surfaces, including P-selectin-adsorbed plain glass, P-selectin-immobilized aldehyde glass substrates (FIG. 9A), and P-selectin immobilized on epoxy glass substrates (FIG. 9B) significantly reduced the microsphere velocities. The microsphere conjugates traveled on PEGylated aldehyde and PEGylated epoxy surfaces without P-selectin at average velocities of 25-30 μm/s and 25-40 μm/s, respectively. The calculated velocity of a microsphere with diameter of 9.95 μm was 57.5 μm/s at a wall shear stress of 0.24 dyn/cm² according to the Goldman's calculation. The velocities of sLe^{x}-bound microspheres on control surfaces velocities were examined each day and used to standardize day-to-day variation in data as plotted in FIGS. 9B and 9D.

After 20 days in PBS at room temperature, P-selectin immobilized surfaces prepared using aldehyde glass substrates lost their adhesiveness, leading to a loss in rolling behavior (FIGS. 9A and 9B). Moreover, there was no significant difference between pre-activated (EDC/NHS) and untreated surfaces in terms of sustained adhesive function. This result can be attributed, it is believed, to unstable chemical bonds involving Schiff bases between aldehydes and PEGs, leading to detachment of P-selectin from the surface over time.

### Epoxy-Based Chemistry

In comparison to aldehyde chemistry, P-selectin covalently immobilized onto epoxy glass exhibited a significant enhancement in long term stability compared to both physisorbed P-selectin and unactivated surfaces (without NHS/EDC treatment) as shown in FIGS. 9C and 9D. After 20 days in PBS at room temperature, pre-activated P-selectin immobilized surfaces exhibited the highest reduction in microsphere velocity (about 40% of controls (microsphere velocity on PEGylated epoxy surfaces without P-selectin)) whereas P-selectin immobilized epoxy glass not treated with EDC/NHS (about 85% of controls) and P-selectin-adsorbed plain glass (about 70% of controls) allowed conjugates to travel relatively faster. After 21 days, the average microsphere velocity was 13.1 μm/s on P-selectin immobilized surfaces compared to 30.6 μm/s on PEGylated surfaces without P-selectin. In some embodiments of the invention, this behavior can be used for surfaces and devices for separating or isolating cells based on rolling behavior, e.g., where specific functionality for extended periods of time is desired.

In the present Example, covalently bound P-selectin on epoxy surfaces was more stable than physisorbed P-selectin. Nevertheless, all of the surfaces tested in the present Example exhibited an increase in microsphere velocity over time, particularly during the first 3 days. This implies that P-selectin immobilization on the surfaces occurs through both covalent binding and physisorption, or that P-selectin forms multi-layers on the surfaces. For example, P-selectin molecules that are adsorbed on top of other P-selectin and/or directly on the surfaces can be readily desorbed from the surfaces for the first few days. After all of the additionally presented P-selectin is desorbed, the observed differences in stability found in the present Example may, it is believed, be attributable to differences between covalent immobilization and physisorption. In some embodiments, actual stability is compared after aging the surfaces for 3 or more days, as adhesive function of covalently bound P-selectin was found in this Example to be substantially constant after that period of time.

In various embodiments, this stabilization process can be sped up by employing, e.g., a flow system for P-selectin immobilization so that additional P-selectin on surfaces can be rapidly removed by shear force.

### Neutrophil Cell Rolling

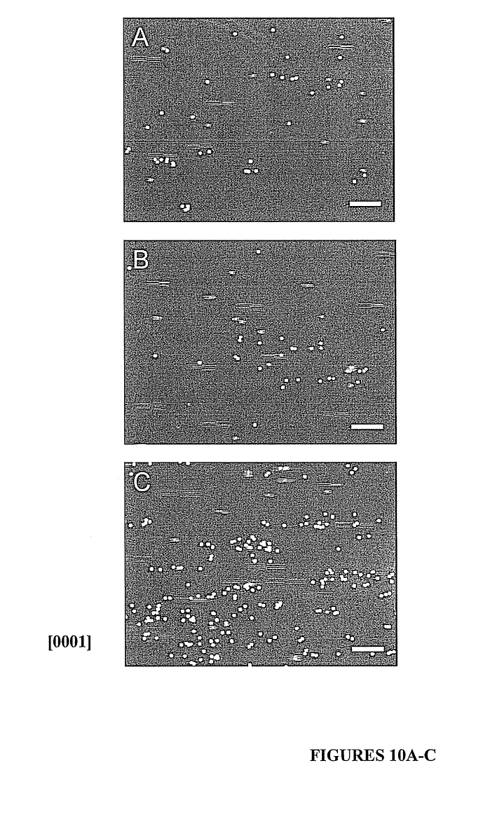

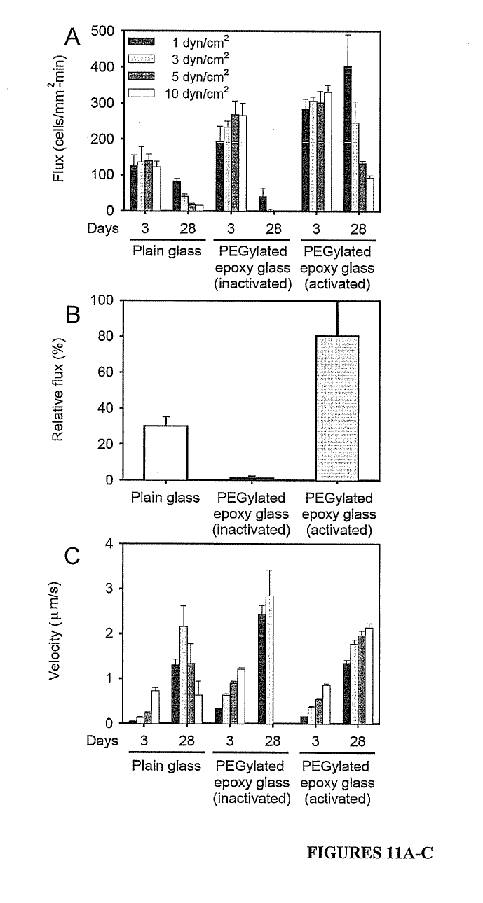

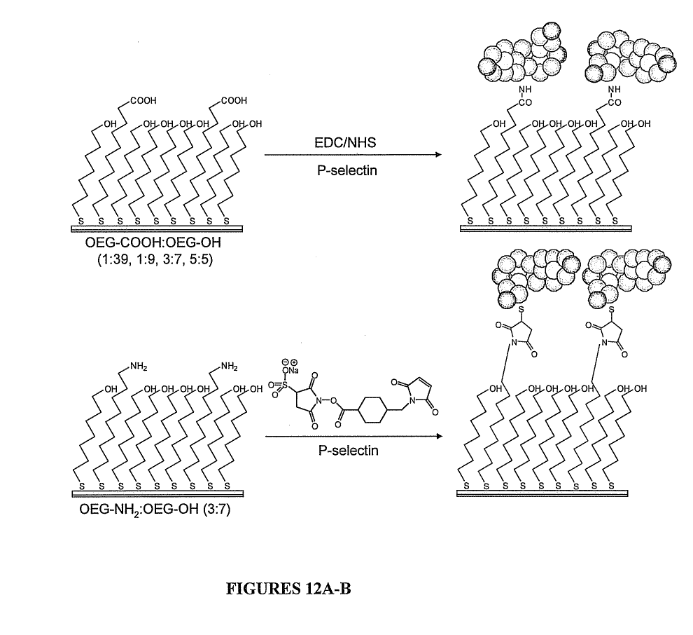

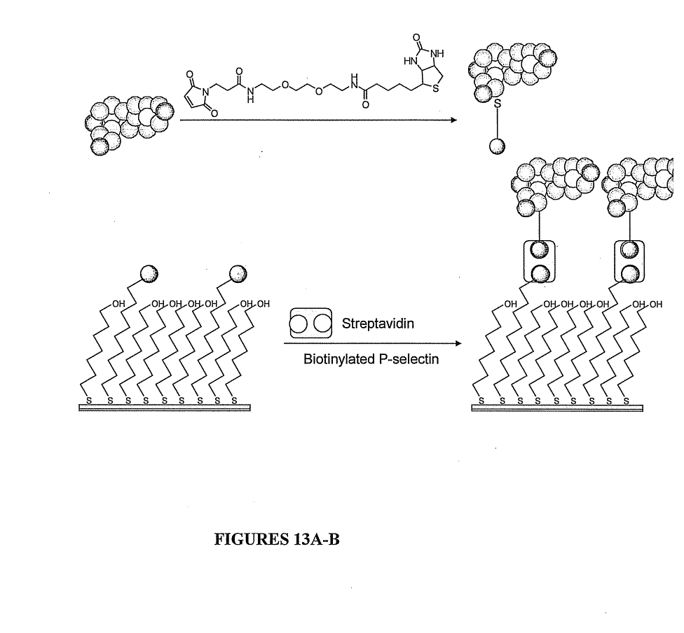

To determine if the microsphere results are consistent with results obtained with live human leukocytes, we investigated neutrophil rolling interaction with immobilized P-selectin using a parallel-plate chamber under flow. Control surfaces which did not have P-selectin (i.e., plain glass and PEGylated epoxy glass slides) showed no cell adhesion. From this in vitro cell rolling assay conducted at four different wall shear stresses (1, 3, 5 and 10 dyn/cm²), the number of rolling cells was significantly greater on P-selectin immobilized surfaces with pre-activation of EDC/NHS than on the rest of the P-selectin-surfaces at 28 days after preparation (FIG. 10). In contrast, rolling fluxes dramatically decreased on older P-selectin-adsorbed surfaces on plain glass and on PEGylated epoxy glass slides without EDC/NHS activation compared with those on newer (3 day-old) surfaces under the same conditions, as shown in FIG. 11A. Specifically, at 3 dyn/cm², rolling fluxes on older P-selectin immobilized on epoxy surfaces (pre-activated with EDC/NHS) did not significantly decrease (80.6±19.1% (mean±SEM) of that on new surfaces), but fluxes on older P-selectin adsorbed glass and on older P-selectin immobilized on epoxy surfaces without EDC/NHS pre-activation dropped to 30.1±5.2% and 1.1±1.1%, respectively (FIG. 11B).

Cell rolling velocity analysis indicates that a large number of neutrophils on aged P-selectin immobilized epoxy surfaces sustain continuous rolling as the shear stress increased, while most cells on the other two surfaces detached and rejoined the free stream (FIG. 11C). The observed rolling velocities of cells were significantly lower than those of microspheres, especially given that shear stresses were higher for cells than for microspheres by an order of magnitude. It is believed, without being held to theory, that this is due to two main differences: (1) the microvilli on the neutrophil surface extend to reconcile the dissociation force applied on the P-selectin-ligand bond, and (2) neutrophils possess the stronger-binding selectin ligand PSGL-1, whereas microspheres are coated with the weaker-binding sLe^{x} group. It is believed, without being held to theory, that the contact area of a neutrophil with a ligand-bearing surface flattens and increases during cell rolling, making additional receptors available for binding. For example, FIGS. 10 and 11 indicate that average rolling velocities of neutrophils on all P-selectin-coated surfaces were lower than those of sLe^{x}-microspheres, although the microspheres traveled at a reduced wall shear stress of 0.24 dyn/cm². In addition, the small number of rolling cells that rolled more slowly on the older P-selectin surface at 5 and 10 dyn/cm² is believed, without being held to theory, to be from small patches of P-selectin retaining their adhesive activity. These data are consistent with data obtained using microspheres, indicating that our pre-screening tests are reliable to quickly test prepared surfaces for adhesive function.

It has been found in the present Example that covalent immobilization of P-selectin enhances cell rolling interactions through improved long-term stability (FIGS. 9, 10, and 11) and homogeneity (FIG. 11) compared to that achieved by typical adsorption protocols. Given the difficulty in cost-effectively isolating large quantities of P-selectin, it is important to note that the immobilization conditions presented here used the same amounts of P-selectin that were used for the adsorbed controls, thus indicating examples of practical utility of the present invention. For example, in some applications, improved stability is typically a requirement for developing implantable devices that capture specific target cell types based on cell rolling.

In some embodiments, the present invention provides methods and surfaces that facilitate optimizing, e.g., the presentation of active P-selectin binding sites. For example, in some embodiments, orientation and density control through chemical immobilization can be used to, e.g., perform controlled studies to uncover the mechanisms of physiological and pathological cell rolling.

### Example 2

### P-Selectin Surface: Mixtures & Orientation

The present Example further provides examples of adjusting the density of P-selectin to provide, e.g., different binding affinities for different cells. Non-fouling surfaces of PEG-based self assembled monolayers (SAMs) were used to prepare surfaces with controlled amounts of reactive sites, and real time observation of binding events with surface plasmon resonance were made. This Example describes a series of quantitative and real-time analyses of P-selectin immobilization and subsequent multivalent effects of microsphere-sLe^{x} conjugates with various diameters monitored by a multi-channel SPR sensor. Through use of NHS/EDC chemistry according to certain embodiments of the present inventions, this Example demonstrates methods for enhancing, and surfaces with enhanced, presentation of P-selectin. To achieve orientation of P-selectin, this Example used thiol chemistry to bind P-selectin to substrates through a cysteine group in the intracellular domain of the P-selectin molecule. Using antibodies to the active site of P-selectin, this Example demonstrates that orientation through thiol chemistry can in some embodiments enhance the availability of P-selectin active sites. In some embodiments, this can be used to enhance and/or control cellular response with covalently immobilized P-selectin surfaces, e.g., in various devices of the present invention.

### Materials and Methods

Recombinant Human P-selectin/Fc chimera (P-selectin) and mouse monoclonal antibody specific for human P-selectin (clone AK-4) were purchased from R&D systems (Minneapolis, Minn.) and used without further purification. Oligo(ethylene glycol) (OEG) alkanethiols with different functional end groups such as HS—(CH₂)₁₁—(O—CH₂CH₂)₄—OH (OEG-OH), HS—(CH₂)₁₁—(O—CH₂CH₂)₆—COOH (OEG-COOH), and HS—(CH₂)₁₁—(O—CH₂CH₂)₆—NH₂ (OEG-NH₂) were purchased from ProChimia (Gdansk, Poland). (HS—(CH₂)₁₀—CONH—(CH₂CH₂—O)₃—(CH₂)₂—NHCO—(CH₂)₄-biotin (OEG-biotin) was provided by Buddy Ratner's group at the University of Washington (Seattle, Wash.). SuperAvidin™-coated microspheres 0.13, 0.51, and 0.97 μm in diameter and multivalent biotinylated Sialyl Lewis(x)-poly(acrylamide) (sLe^{x}-PAA-biotin) were supplied by Bangs Laboratories (Fishers, Ind.) and Glycotech (Gaithersburg, Md.), respectively. All other chemicals were obtained from Sigma-Aldrich (St. Louis, Mo.) unless otherwise specified.

### Surface Plasmon Resonance (SPR) Sensor

A custom-built SPR sensor with 4 channels developed at the Radio Institute of Engineering and Electronics, Academy of Sciences (Prague, Czech Republic) was used in this study. The SPR sensor is based on the Kretschmann geometry of the attenuated total reflection (ATR) method and wavelength interrogation Briefly, a functionalized SPR chip was attached to the base of an optical prism from the glass side mediated with a refractive index matching fluid (Cargille Labs, Cedar Groves, N.J.). The metal side was mechanically pressed against an acrylic flow cell with a laser cut 50 μm thick Mylar gasket and each channel was connected to a multichannel peristaltic pump, creating 4 channels. The excitation of the surface plasmon is accompanied by the transfer of optical energy into surface plasmon and dissipation in the metal layer, resulting in a narrow dip in the spectrum of reflected light. The wavelength at which the resonant excitation of the surface plasmon occurs depends on the refractive index of the analyte in proximity to the SPR surface. As the refractive index increases, the resonant wavelength shifts to high surface concentration (mass per unit area). Thus, an SPR sensorgram is a plot of resonant wavelength shift versus time, giving the amount of analyte binding as a function of time.

### Preparation of SAMs on SPR Sensor Chip Surfaces

Glass chips were coated with a 2 nm adhesion-promoting chromium film, followed by a 50 nm surface plasmon-active-layer of gold by electron beam evaporation as illustrated in FIGS. 12A and 12B. The gold surface was cleaned before subsequent formation of mixed SAMs by washing with absolute ethanol and drying by nitrogen blowing. Organic contaminants were then removed by an UV ozone cleaner for 20 minutes, finished by washing the surface with 18.2 MΩ·cm deionized water and absolute ethanol. The surface was dried under nitrogen flow before further functionalization.

SAMs were formed by soaking gold coated substrates in a solution containing 100 μM total OEG-alkanethiol concentration in ethanol at room temperature overnight. The following mixtures of different OEG-alkanethiols were used at the indicated molar ratios: OEG-COOH:OEG-OH (1:39, 1:9, 3:7, 5:5), OEG-NH₂:OEG-OH (3:7), and OEG-biotin:OEG-OH (1:9). All SAMs were then rinsed extensively with water and ethanol, followed by drying in a stream of nitrogen. All buffers and solutions were degassed under vacuum for 30 minutes before being introduced into the SPR system.

### P-selectin Immobilization on the SAMs

P-selectin was immobilized onto surfaces of the SAMs as follows. The chemistry used for mixed SAMs of OEG-COOH/OEG-OH is illustrated in FIG. 12A. 10 mM phosphate buffer (PB) was first flowed into a chip at a flow rate of 50 μL/min for 5 min. A 1:1 (v/v) mixture of 1-ethyl-3-(3-dimethylaminopropyl)-carbodiimide (EDC) at 76.68 mg/mL and N-hydroxysuccinimide (NHS) at 11.51 mg/mL was injected to activate carboxyl groups on the SAMs for 10 minutes. After flowing for 5 minutes, P-selectin (20 μg/mL in PB) was injected and flowed to be immobilized for 7 minutes. The chip surface was then washed with PB for 5 minutes, followed by ethanolamine (100 mM in PB) to inactivate remaining active ester groups and to remove loosely bound P-selectin from the surface. To compare this covalent immobilization with physisorption, some channels were used as a reference channel wherein P-selectin was adsorbed on the surface without EDC/NHS activation. Both covalently immobilized and physisorbed surfaces were washed with 150 mM Tris-HCl buffered saline (TBS) to compare surface stability. To control density of immobilized P-selectin, mixed SAMs of OEG-COOH/OEG-OH at different ratios were used and P-selectin was immobilized under the same condition described above.

For mixed SAMs of OEG-NH₂:OEG-OH, the surface was first immersed in a solution of sulfosuccinimidyl 4-(N-maleimidomethyl)cyclohexane-1-carboxylate (sulfo-SMCC, Pierce, Rockford, Ill.) at room temperature for 1 hour to convert amine groups to maleimide groups that specifically binds to a cysteine residue in P-selectin. The chip was mounted on the SPR sensor and PB and PBS were sequentially flowed into the channels. P-selectin was immobilized under the same condition, followed by the same washing steps (FIG. 12B). To investigate the specificity of the reaction, 500 μg (>50 times more than the amount of P-selectin flowed) of bovine serum albumin (BSA) was flowed into a channel.

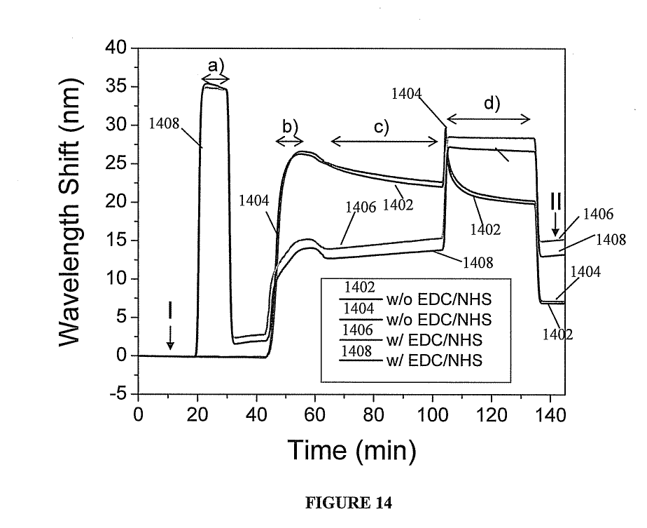

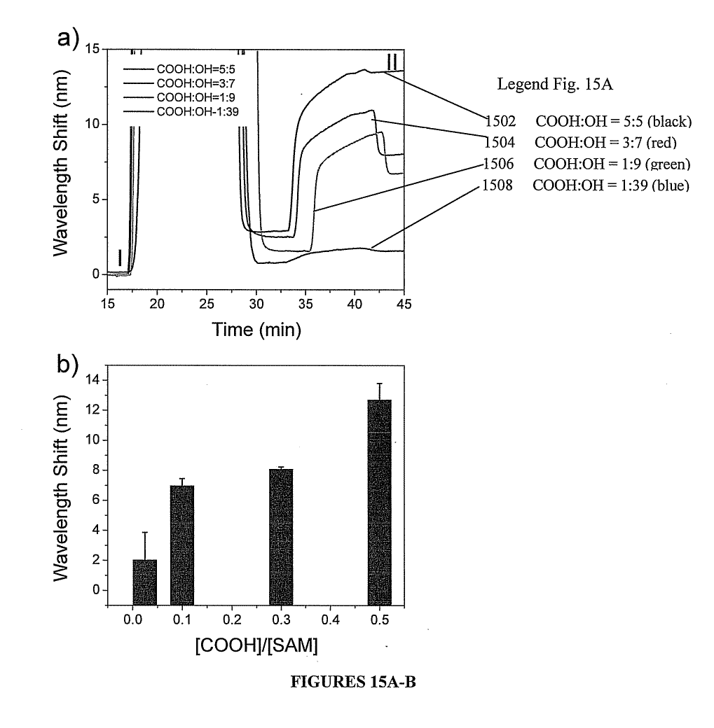

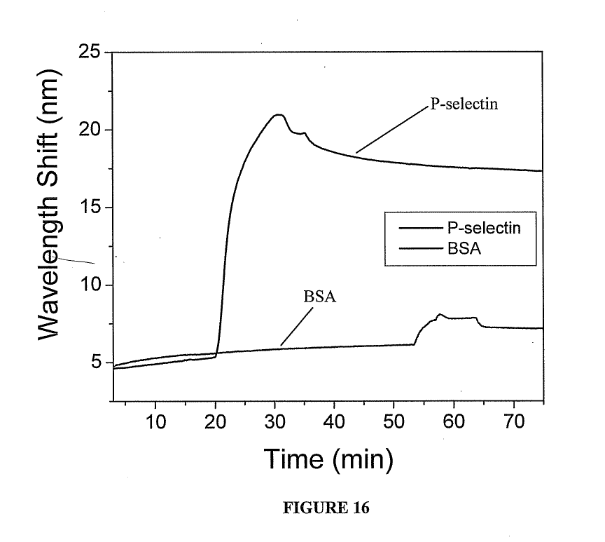

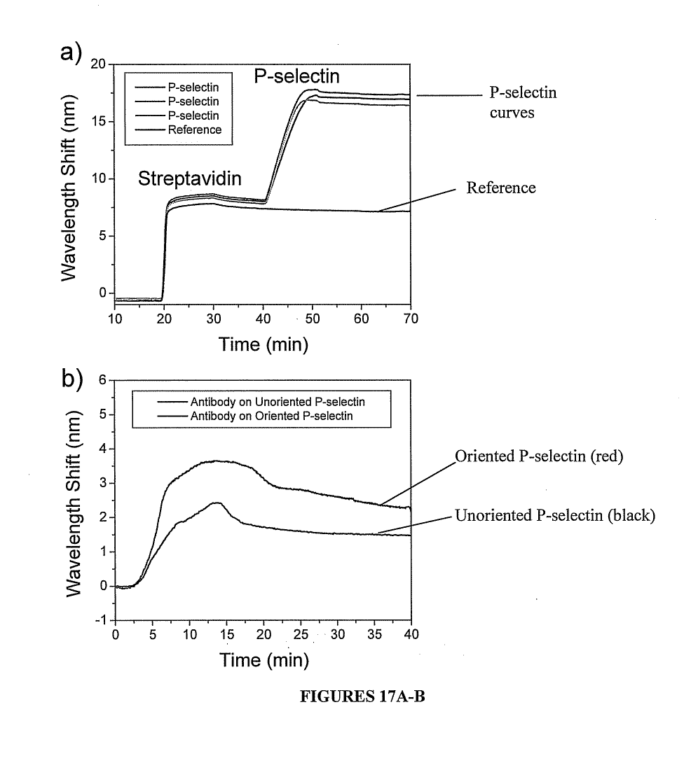

For P-selectin immobilization on mixed SAMs of OEG-biotin/OEG-OH, P-selectin was biotinylated using maleimide-PEO₂-biotin (Pierce, Rockford, Ill.) before SPR measurement as shown in FIG. 13A. A solution of P-selectin at 50 μL of 1 mg/mL P-selectin in PBS was mixed with 50 molar excess maleimide-PEG₂-biotin solution at 4° C. overnight. The reaction mixture was purified by 4 cycles of ultrafiltration using a 10K molecular weight cut-off membrane. Each cycle was performed at 14,000×g for 30 minutes. The mixed SAM of OEG-biotin/OEG-OH was mounted on the SPR device and 10 μg/mL streptavidin in PBS was flowed for 10 minutes to create binding sites for the biotinylated P-selectin. P-selectin was then immobilized under the same condition used for other mixed SAM surfaces via strong biotin/avidin binding (FIG. 13B). All immobilization in the SPR device was carried out at a flow rate of 50 μL/min. Further data from this Example are illustrated in FIGS. 14-17B.

### Example 3

### Surface with Adhesion Moiety and Biologically Active Agent

The following Example describes embodiments of the invention that use P-selectin as an adhesion moiety and TRAIL as a biologically active agent (e.g. ligand). It is to be understood that the present inventions are not limited to these specific moieties and agents.

### Covalent Immobilization of P-Selectin on Substrates in 2-D.

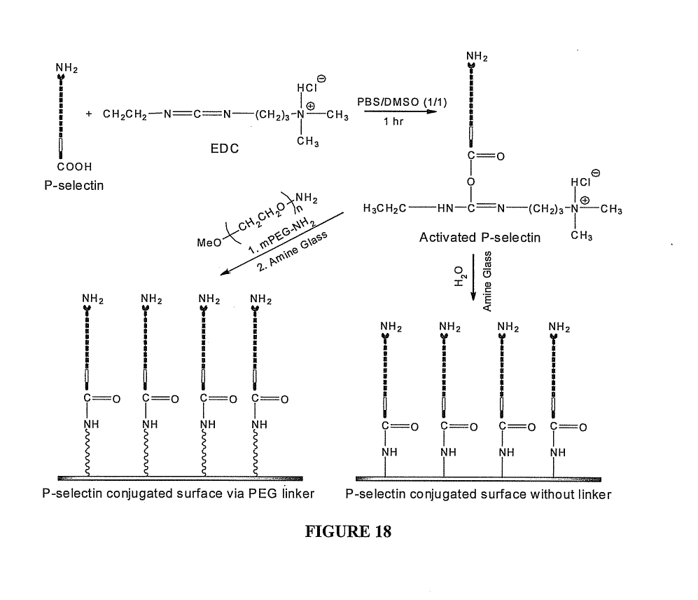

In some embodiments of the present invention, N-termini of P-selectin are oriented so that the protein has selectivity for P-selectin glycoprotein ligand-1 (PSGL-1) which is a disulfide-bonded, homodimeric mucin (approximately 250 kDa) present on leukocytes. In some embodiments, the carboxylic ends of the protein are utilized for covalent conjugation with a substrate material. For example, prior to covalent conjugation of P-selectin with desired substrates (glass or polymers), carboxylic end groups (γ-carboxylic acid) of P-selectin can be pre-activated by 1-[3-(dimethylamino)propy1]-3-ethylcarbodiimide/HCl (EDC) (schematically illustrated in FIG. 18). The activated form of proteins induced by EDC treatment has been used in covalent incorporation of biologically active molecules to polymers.

This Example describes two methods to achieve covalent immobilization of P-selectin on substrate materials.

Immobilization of P-Selectin by Direct Conjugation with Glass Substrate

The EDC-pre-activated carboxyl group of P-selectin is covalently conjugated to the amine glass substrate in aqueous solution (see, e.g., FIG. 18). Different densities of P-selectin can be formed on the surface by application of pre-activated P-selectin at different concentrations. Bovine serum albumin (BSA) can be employed to reduce non-specific binding and as a part of routine rinsing steps. Non-covalently or loosely bound P-selectin can be washed out using 1 M ethanolamine in water (pH 8.5). Non-specifically adsorbed protein can be removed by brief sonication in NaCl-supplemented buffer with 0.05-0.1% Tween-20, followed by rinsing with phosphate buffered saline (PBS). These rinsing steps can be performed, e.g., as the final step for all methods of this Example.

### Immobilization of P-Selectin by Conjugation Through PEG Linkers

PEG is non-toxic, non-immunogenic, non-antigenic, and FDA approved. Although most PEGylation methods have utilized a target protein's amine groups, in this Example, PEG is conjugated via the carboxylic ends of P-selectin so that selectivity of the protein remains intact. Amine terminated monofunctional PEG linkers can provide reactive sites (primary amine groups) for the pre-activated carboxylic ends of human P-selectin, resulting in covalent conjugation between P-selectin and mPEG (see, e.g., FIG. 18). The P-selectin/mPEG conjugates can then be immobilized on glass via methoxy groups or modification of mPEG using silanization. In some embodiments, this step can be used to orient P-selectin as well as to reduce non-specific protein adsorption on the surface.

### Covalent Immobilization of P-Selectin in 3-D

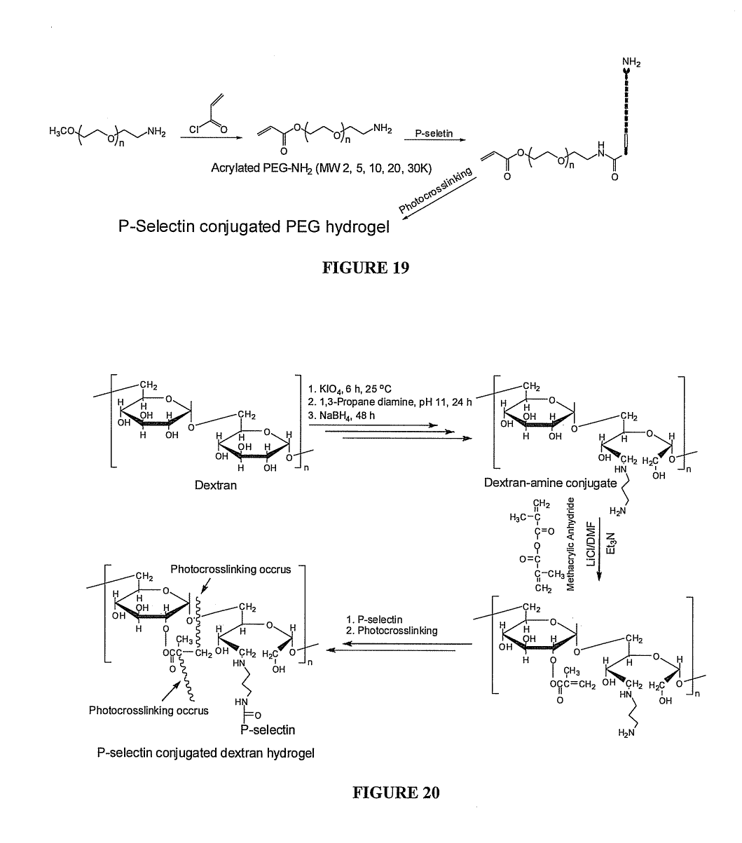

In some embodiments of the invention, two-dimensional (2-D) structures generated by inventive methods can be translated to develop three-dimensional (3-D) matrices as schematically illustrated, e.g., in FIG. 19 and as described below.

### PEG Hydrogel as a 3-D Matrix

Methoxy groups on mPEG-NH₂ can be chemically changed to acrylic groups, enabling the PEG to be photocrosslinkable. Acrylated PEG-NH₂ is covalently conjugated to P-selectin using substantially the same chemistry depicted in FIG. 19. The acrylated PEG/P-selectin conjugates can then be photocrosslinked by photoinitiators such as, e.g., 2,2-dimethoxy-2-phenyl-acetophenone. The degree of crosslinking and density of conjugated P-selectin can be controlled using different amounts of acryloyl chloride and/or different molecular weights of PEG-NH₂.

### Dextran Hydrogel as a 3-D Matrix

Polycationic polymers such as poly-L-lysine, polyethylenimine, and dextran derivatives have been commonly used as platforms for biomedical applications including non-viral gene delivery. A number of biomolecules (e.g., RGD peptides and folate) have been also conjugated to these polymers using a variety of conjugation chemistries in order to provide desired biological functions (such as selectivity) to the polymers. Nevertheless, polycationic polymers exhibit toxic effects both in vitro and in vivo attributed to their non-specific electrostatic interactions with biological substances, and consequently clinical trials of the polymers have been retarded. Among the polymers, dextran derivatives have shown minimal toxicity due to their low charge density and excellent biocompatibility. Further, the polymer can be formed as a 3-D hydrogel by incorporating acryl groups into the polymer backbone. In some embodiments, dextran is employed as a material to construct a 3-D structure. The primary amine groups that are capable of being conjugated with P-selectin can be introduced to the dextran matrix using methacrylic anhydride as schematically illustrated in FIG. 20. In some embodiments, acryl groups are incorporated to create photocrosslinkable moieties in the polymer backbone, followed by P-selectin conjugation via primary amine groups using substantially the same chemistry as illustrated earlier in this Example. The P-selectin/dextran conjugates can be photocrosslinked to form a 3-D structure hydrogel. In some embodiments, a dextran hydrogel containing P-selectin can have increased water solubility over, e.g., a PEG hydrogel. In some embodiments, the dextran/P-selectin hydrogel can exhibit greater degradability than a PEG hydrogel, resulting in exposure of fresh P-selectin on the surface.

### Covalent Conjugation of TRAIL (aka APO2L)

TRAIL conjugation can be used in conjunction with P-selectin conjugation described above. The N-termini of TRAIL can be used as conjugation sites and are compatible with conjugation chemistries such as NHS/EDC chemistry. TRAIL can be first conjugated to mPEG-NHS and the conjugates can be immobilized on a glass substrate as described above in this Example. In some embodiments, this approach can be used to achieve surface functionalization in 2-D using a chemistry similar to that used for P-selectin conjugation.

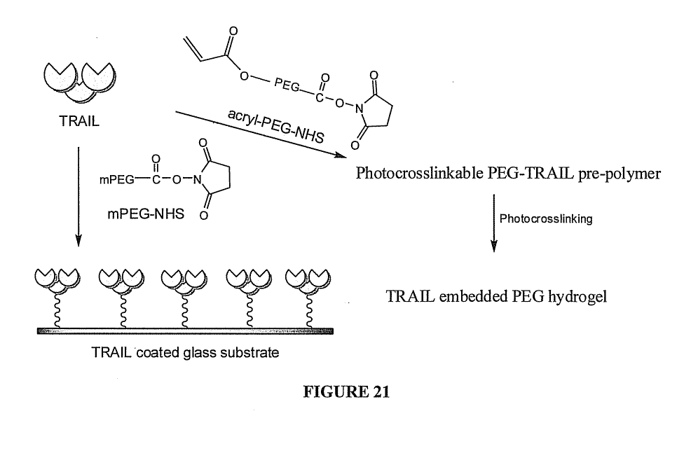

TRAIL can be conjugated to acryl-PEG-NHS. The resulting acryl-PEG-TRAIL conjugates can be photocrosslinked, resulting in TRAIL embedded in a PEG hydrogel 3-D structure. Dextran-based 3-D hydrogel containing TRAIL can be prepared using substantially the same chemistry depicted in FIG. 19. Synthetic routes for TRAIL conjugation for both 2-D and 3-D structures are schematically illustrated in FIG. 21.

In some embodiments, methods of the present Example can provide biologically multifunctional substrates. For example, using the above approaches for P-selectin conjugation as well as TRAIL conjugation, both proteins can be covalently immobilized onto the same substrate.

### Further Introduction of Biological Functions

For cell rolling, P-selectin can potentially be replaced or co-immobilized with α4 integrins that induce selectin-independent rolling of hematopoietic progenitor cells. By controlling density of these cell rolling-inducing molecules and/or co-immobilizing one or more other adhesive moieties (e.g., RGD peptides, folate, and EGF) materials with different specificity to different target cells can be provided by some embodiments of the invention.

For example, for metastatic cancer treatment, one or more other anticancer drugs such as methotrexate, Taxol, and/or Doxorubicin can be attached instead of or along with TRAIL on the surface so that a “cocktail” therapy can be achieved that may efficiently induce apoptosis of tumor cells.

Other materials that can serve as suitable linkers include, but are not limited to, surface modified polycationic polymers such as polylysines, polyethylenimines, and polyamidoamine (PAMAM) dendrimers. For example, primary amine groups on PAMAM dendrimers can be used for covalent conjugations with selectins and TRAIL as well as other targeting molecules and chemotherapeutic drugs. Remaining amine groups can be altered to carboxylate groups using succinic anhydride. Carboxyl groups may be conjugated to the surface (aminated glass for 2-D or amine-PEG-acryl for 3-D). In some embodiments, carboxylate groups are used to reduce non-specific protein adsorption.

### Examples 4-7

### Devices

It is to be understood that a wide variety of devices can be fabricated using materials and surfaces provided by certain embodiments of the present invention. Examples 4-7 provide several non-limiting examples of such devices.

### Example 4

### Killer Stents

In some embodiments of the invention, inventive materials and/or surfaces are used to make implantable devices to capture and kill metastatic cancer cells in the bloodstream. Over 1.5 million people in North America and over 11 million people worldwide are diagnosed with new cases of cancer each year. About 20% of these people will develop metastatic masses as complications. The formation of secondary tumors can be hindered and/or prevented using a coated killer stent that selectively captures and kills cancer cells before they engraft.

### Example 5

### Donor Stents

In some embodiments of the invention, inventive materials and/or surfaces are used to make implantable devices to isolate stem cells from a matched donor for use in bone marrow transplants. About 35,000 people in the US develop leukemia each year. Typically, high doses of chemotherapy drugs are used to kill cancerous cells. Unfortunately, bone marrow cells that regenerate blood cells of various lineages are also killed in this toxic process. Bone marrow transplants are part of standard post chemotherapy treatments to restore normal blood function, but the availability of suitable and willing donors severely limits the use of this treatment.

In some embodiments of the invention, an implantable device to isolate bone marrow-derived stem cells from the circulating bloodstream is provided. Such a device could radically reduce donor burden and trauma. This device can potentially enlarge significantly the number of willing donors, thus allowing marrow transplant treatments to be available to a much larger number of patients.

### Example 6

### Healing Stents

In some embodiments of the invention, inventive materials and/or surfaces are used to make implantable devices to capture adult stem cells circulating in the bloodstream and direct them to an area in need of regeneration. There are over half a million heart attack survivors each year in the US in need of heart tissue regeneration. Nearly one and a half million people suffer osteoporosis-related fractures each year in the US, with 70,000 deaths from complications. In some embodiments of the invention, an implantable device for stimulating and trafficking a patient's own stem cells to facilitate healing is provided.

### Example 7

### Blood Disposables

In some embodiments of the invention, inventive materials and/or surfaces are used to make disposable modules to isolate stem cells from donated blood. Over 14 million liters of blood are donated in the US annually. In some embodiments, stem cells are harvested during blood donation using isolation modules (e.g., plastic, disposable modules) comprising stem cell-targeted cell rolling materials and/or surfaces provided by some embodiments of the present invention. For example, pooling and expansion of stem cells harvested with such modules could enable reducing costs of cell supplies for a range of blood disorders.

### Example 8

### Further Control Experiments

In this Example, a series of control experiments were conducted to confirm that observed cell rolling responses were due to specific interactions between sLe^{x} and P-selectin. Microspheres without ligands (sLe^{x}) demonstrated no rolling behavior (average velocities of 32-40 μm/s on the P-selectin coated surfaces) and surfaces without P-selectin did not reduce velocities of flowing microsphere conjugates. Data on the results of these experiments are presented in FIGS. 22A and 22B. FIG. 22A presents data on the measured velocities of native microspheres (without sLe^{x}) on control (plain+BSA) and various experimental surfaces. The measured velocities were not significantly different, indicating that there is minimal non-specific interaction between substrates and microspheres. FIG. 22B presents data on the comparison of velocities of microsphere-sLe^{x} conjugates on P-selectin immobilized substrates before and after treatment with an antibody for P-selectin. It was observed that velocities significantly increased when substrates were preincubated with antibody, indicating that the reduced velocities observed in experimental groups were due to a direct interaction of P-selectin with sLe^{x}.

In addition, to block specific interaction between P-selectin and sLe^{x} on microspheres, P-selectin coated surfaces were post-treated using P-selectin antibody, followed by perfusion of microsphere conjugates into the flow chamber. After antibody treatment, the microsphere average velocities on P-selectin-coated surfaces were increased from 0.4 to 31.6 μm/s and 3.4 to 29.2 μm/s on P-selectin immobilized epoxy and aldehyde surfaces, respectively (see, e.g., FIG. 22B). These results indicate that the observed velocity reduction on P-selectin-coated surfaces is due to a P-selectin-mediated interaction.

Examples of fluorescence microscopy images of P-selectin antibody-FITC conjugate of this Example are shown in FIGS. 23A-D. P-selectin antibody-FITC conjugate was incubated on untreated amine glass and amine glass substrates (FIG. 23A) with 5 μg (FIG. 23B); 10 μg (FIG. 23C), and 20 μg (FIG. 23D) of P-selectin. P-selectin was immobilized onto amine glass overnight after pre-activation with EDC and NHS. The antibody-FITC conjugate was incubated for 2 hours. Significant aggregation of P-selectin was observed with this chemistry. Images were taken using a 10× objective.

### Other Embodiments

All literature and similar material cited in this application, including, but not limited to, patents, patent applications, articles, books, treatises, and web pages, regardless of the format of such literature and similar materials, are expressly incorporated by reference in their entirety for all purposes. In the event that one or more of the incorporated literature and similar materials differs from or contradicts this application in aspects including, but not limited to, defined terms, term usage, described techniques, and/or the like, this application controls.

The section headings used herein are for organizational purposes only and are not to be construed as limiting the subject matter described in any way.

While the present invention has been described in conjunction with various embodiments and examples, it is not intended that the present invention be limited to such embodiments or examples. On the contrary, the present inventions encompass various alternatives, modifications, and equivalents, as will be appreciated by those of skill in the art.

The invention should not be read as limited to the described order or elements unless stated to that effect. It should be understood that various changes in form and detail may be made without departing from the scope of the present invention. Therefore, all embodiments that come within the scope and spirit of the present invention and equivalents thereof are claimed.

What is claimed is:

1. A method for inducing cell rolling comprising contacting a cell with a substrate surface, wherein the surface is at least partially coated with an ordered layer of cell adhesion molecules that are bound to the surface through a covalent bond, and wherein the cell comprises a moiety on its surface that is recognized by the cell adhesion molecules.

2. The method of claim 1, wherein the cell adhesion molecules are bound to the surface through interactions that are entirely covalent.

3. The method of claim 1, wherein the cell adhesion molecules are bound to the surface through interactions that include one or more non-covalent bonds.

4. The method of claim 1, wherein the density of the cell adhesion molecules in the ordered layer is substantially uniform.

5. The method of claim 1, wherein the orientation of the cell adhesion molecules in the ordered layer is substantially uniform.

6. The method of claim 1, wherein the ordered layer comprises a patternwise distribution of the cell adhesion molecules.

7. The method of claim 1, wherein the ordered layer comprises a patternwise density of the cell adhesion molecules.

8. The method of claim 1, wherein the ordered layer comprises a patternwise orientation of the cell adhesion molecules.

9. The method of claim 1, wherein the velocity of cell rolling over the ordered layer is substantially proportional to the shear stress applied to the ordered layer.

10. The method of claim 1, wherein cell rolling over the ordered layer can be observed at least 3 days after the surface was coated.

11. The method of claim 1, wherein cell rolling over the ordered layer can be observed at least 5 days after the surface was coated.

12. The method of claim 1, wherein cell rolling over the ordered layer can be observed at least 10 days after the surface was coated.

13. The method of claim 1, wherein cell rolling over the ordered layer can be observed at least 15 days after the surface was coated.

14. The method of claim 1, wherein cell rolling over the ordered layer can be observed at least 20 days after the surface was coated.

15. The method of claim 1, wherein cell rolling over the ordered layer can be observed at least 25 days after the surface was coated.

16. The method of claim 1, wherein cell rolling over the ordered layer can be observed at least 28 days after the surface was coated.

17. The method of claim 1, wherein the ordered layer comprises a density of cell adhesion molecules between about 10 ng/cm² and about 600 ng/cm².

18. The method of claim 1, wherein the ordered layer comprises a density of cell adhesion molecules greater than about 30 ng/cm².

19. The method of claim 17, wherein the ordered layer comprises a density of cell adhesion molecules between about 30 ng/cm² to about 360 ng/cm².

20. The method of claim 19, wherein the ordered layer comprises a density of cell adhesion molecules between about 50 ng/cm² to about 300 ng/cm².

21. The method of claim 20, wherein the ordered layer comprises a density of cell adhesion molecules between about 100 ng/cm² to about 200 ng/cm².

22. The method of claim 1, wherein the cell adhesion molecules have a dissociation constant (K_{D}) for interaction with the moiety on the surface of the cell that is greater than about 1×10⁻⁸ M.

23. The method of claim 22, wherein the dissociation constant (K_{D}) is in the range of about 1×10⁻⁴ M to about 1×10⁻⁷ M, inclusive.

24. The method of claim 1, wherein the cell adhesion molecules are selected from the group consisting of selectins, integrins, cadherins, immunoglobulin cell adhesion molecules, and combinations thereof.

25. The method of claim 1, wherein the cell adhesion molecules are selected from the group consisting of E-selectin, P-selectin, L-selectin, and combinations thereof.

26. The method of claim 25, wherein the cell adhesion molecule is a selectin that is responsible for localization of metastatic cancer cells.

27. The method of claim 1, wherein the cell adhesion molecules comprise P-selectin.

28. The method of claim 1, wherein the cell adhesion molecules comprise integrin ITGA4.

29. The method of claim 1, wherein the cell adhesion molecules are selected from the group consisting of E-cadherin, N-cadherin, P-cadherin, and combinations thereof.

30. The method of claim 1, wherein the cell adhesion molecules are selected from the group consisting of neural cell adhesion molecules, intracellular adhesion molecules, vascular cell adhesion molecules, platelet-endothelial cell adhesion molecules, L1 cell adhesion molecules, and combinations thereof.

31. The method of claim 1, wherein the cell adhesion molecules are selected from the group consisting of aptamers, carbohydrates, and peptides.

32. The method of claim 1, wherein the cell adhesion molecules comprise one or more extracellular matrix cell adhesion molecules.

33. The method of claim 32, wherein the cell adhesion molecules are selected from the group consisting of vitronectin, fibronectin, and laminin.

34. The method of claim 1, wherein the cell adhesion molecules are covalently bound to the surface via an epoxy group.

35. The method of claim 1, wherein the cell adhesion molecules are covalently bound to the surface via a group selected from the group consisting of amine groups, aldehyde groups, and combinations thereof.

36. The method of claim 1, wherein the cell adhesion molecules are covalently bound to the surface via a group selected from the group consisting of vinyl groups, thiol groups, carboxylate groups, and hydroxyl groups.

37. The method of claim 1, wherein the cell adhesion molecules are covalently bound to the surface via a linker moiety.

38. The method of claim 37, wherein the linker moiety is covalently bound to the cell adhesion molecule and to the surface.

39. The method of claim 37, wherein the linker moiety is non-covalently bound to the cell adhesion molecule and covalently bound to the surface.

40. The method of claim 39, wherein the linker moiety is bound to the cell adhesion molecule via a non-covalent ligand/receptor pair interaction.

41. The method of claim 37, wherein the linker moiety comprises one or more moieties selected from the group consisting of dextrans, dendrimers, polyethylene glycol, poly(L-lysine), poly(L-glutamic acid), poly(D-lysine), poly(D-glutamic acid), polyvinyl alcohol, polyethylenimine, and combinations thereof.

42. The method of claim 1, wherein the substrate further comprises one or more cell modifying ligands.

43. The method of claim 42, wherein the cell modifying ligand is targeted to a specific cell type.

44. The method of claim 43, wherein the specific cell type is a cancer cell.

45. The method of claim 43, wherein the specific cell type is a stem cell.

46. The method of claim 42, wherein the cell modifying ligands are bound to the surface through a covalent bond.

47. The method of claim 42, wherein the cell modifying ligands comprise at least one cell modifying ligand that is covalently attached to the surface and at least one cell modifying ligand that is non-covalently attached to the surface.

48. The method of claim 42, wherein the cell modifying ligands disrupt cellular function in cancer cells.

49. The method of claim 44, wherein the cell modifying ligands comprise tumor necrosis factor (TNF)-related apoptosis inducing ligand (TRAIL).

50. The method of claim 45, wherein the cell modifying ligands comprise ligands selected from the group consisting of basic fibroblast growth factor 2 (FGF-2), bone morphogenic protein 2 (BMP-2), and combinations thereof.

51. The method of claim 42, wherein the cell modifying ligands disrupt or induce one or more processes selected from the group consisting of cell quiescence, cell proliferation, cell migration, cell de-differentiation, cell spreading, cell attachment, and cell differentiation.

52. The method of claim 1, wherein the substrate is an intravascular stent.

53. The method of claim 1, wherein the substrate is a vascular graft.

54. The method of claim 1, wherein the substrate comprises a glass.

55. The method of claim 1, wherein the substrate comprises an implantable and/or injectable material.

56. The method of claim 55, wherein the substrate comprises an injectable polymer.

57. The method of claim 55, wherein the implantable and/or injectable material stimulates cells to produce a cell modifying ligand.

58. The method of claim 55, wherein the substrate is implanted into a site that serves as a niche environment for stimulating vascularization.

59. The method of claim 1, wherein the substrate comprises a porous polymeric matrix and the surface comprises the surface of the pores.

60. The method of claim 27, wherein P-selectin is linked to the surface via its C-terminus.

61. The method of claim 27, wherein P-selectin is linked to the surface via a cysteine residue in the intracellular domain of P-selectin.

62. The method of claim 1, wherein the substrate is substantially degradable.

63. The method of claim 62, wherein the substantially degradable substrate comprises at least one surface-erodible polymer.

64. The method of claim 63, wherein the surface erodible polymer is selected from the group consisting of poly(glycerol sebacic acid), polyanhydrides, poly(diol citrates), and combinations thereof.

65. The method of claim 62, wherein the substrate comprises a hydrogel material.

66. The method of claim 65, wherein the hydrogel material is selected from the group consisting of poly(ethylene glycol), hyaluronic acid, and combinations thereof.

67. The method of claim 62, wherein the substrate further comprises one or more cell modifying ligands.

68. The method of claim 67, wherein the cell modifying ligands are exposed as the substrate degrades.

69. The method of claim 1, wherein the substrate comprises entrapped cell modifying ligands.

70. The method of claim 69, wherein the entrapped cell modifying ligands are entrapped within releasing vehicles.

71. The method of claim 70, wherein the releasing vehicles are selected from the group consisting of nanoparticles, microparticles, and combinations thereof.

72. The method of claim 70, wherein cell modifying ligands released from the releasing vehicles are transported to the surface of the substrate.

73. The method of claim 62, wherein the substrate further comprises particles or regions of more slowly degrading materials that contain entrapped and/or surface-bound cell modifying ligands.

74. The method of claim 73, wherein particles or regions containing cell modifying ligands are exposed as the substrate degrades.

75. The method of claim 1, wherein the cell rolls over the ordered layer but does not stop.

76. The method of claim 1, wherein the ordered layer of cell adhesion molecules further comprises antibodies.

77. The method of claim 76, wherein the antibodies facilitate stopping cells that roll over the ordered layer.

78. The method of claim 1, wherein the cell invades the substrate and becomes entrapped.

79. The method of claim 78, wherein the entrapped cell is a circulating cell.

80. The method of claim 79, wherein the circulating cell is selected from the group consisting of metastasizing cancer cells, stem cells, progenitor cells, and combinations thereof.

81. The method of claim 80, wherein the circulating cell is an endothelial progenitor cell.

82. The method of claim 1, wherein the substrate comprises a prefabricated vascularized matrix.

83. The method of claim 82, wherein the vascularized matrix is implantable.

84. The method of claim 82, wherein the vascularized matrix is created with endothelial cells from a patient and the substrate is administered to the patient.

85. A substrate comprising a surface, wherein the surface is at least partially coated with an ordered layer of selectin molecules that are bound to the surface through a covalent bond, wherein the substrate induces cell rolling of a cell that comprises a moiety on its surface that is recognized by the selectin molecules.

86. The substrate of claim 85, wherein the selectin molecules are bound to the surface through interactions that are entirely covalent.

87. The substrate of claim 85, wherein the selectin molecules are bound to the surface through interactions that include one or more non-covalent bonds.

88. The substrate of claim 85, wherein the density of the selectin molecules in the ordered layer is substantially uniform.

89. The substrate of claim 85, wherein the orientation of the selectin molecules in the ordered layer is substantially uniform.

90. The substrate of claim 85, wherein the ordered layer comprises a patternwise distribution of the selectin molecules.

91. The substrate of claim 85, wherein the ordered layer comprises a patternwise density of the selectin molecules.

92. The substrate of claim 85, wherein the ordered layer comprises a patternwise orientation of the selectin molecules.

93. The substrate of claim 85, wherein the velocity of cell rolling over the ordered layer is substantially proportional to the shear stress applied to the ordered layer.

94. The substrate of claim 85, wherein cell rolling over the ordered layer can be observed at least 3 days after the surface was coated.

95. The substrate of claim 85, wherein cell rolling over the ordered layer can be observed at least 5 days after the surface was coated.

96. The substrate of claim 85, wherein cell rolling over the ordered layer can be observed at least 10 days after the surface was coated.

97. The substrate of claim 85, wherein cell rolling over the ordered layer can be observed at least 15 days after the surface was coated.

98. The substrate of claim 85, wherein cell rolling over the ordered layer can be observed at least 20 days after the surface was coated.

99. The substrate of claim 85, wherein cell rolling over the ordered layer can be observed at least 25 days after the surface was coated.

100. The substrate of claim 85, wherein cell rolling over the ordered layer can be observed at least 28 days after the surface was coated.

101. The substrate of claim 85, wherein the ordered layer comprises a density of selectin molecules between about 10 ng/cm² and about 600 ng/cm².

102. The substrate of claim 85, wherein the ordered layer comprises a density of selectin molecules greater than about 30 ng/cm².

103. The substrate of claim 101, wherein the ordered layer comprises a density of selectin molecules between about 30 ng/cm² to about 360 ng/cm².

104. The substrate of claim 103, wherein the ordered layer comprises a density of selectin molecules between about 50 ng/cm² to about 300 ng/cm².

105. The substrate of claim 104, wherein the ordered layer comprises a density of selectin molecules between about 100 ng/cm² to about 200 ng/cm².

106. The substrate of claim 85, wherein the selectin molecules have a dissociation constant (K_{D}) for interaction with a moiety on the surface of the cell that is greater than about 1×10⁻⁸ M.

107. The substrate of claim 106, wherein the dissociation constant (K_{D}) is in the range of about 1×10⁻⁴ M to about 1×10⁻⁷ M, inclusive.

108. The substrate of claim 85, wherein the selectin molecules comprise a selectin selected from the group consisting of E-selectin, P-selectin, L-selectin, and combinations thereof.

109. The substrate of claim 108, wherein the selectin molecules comprise P-selectin.

110. The substrate of claim 85, wherein the selectin molecules comprise a selectin that is responsible for localization of metastatic cancer cells.

111. The substrate of claim 85, wherein the selectin molecules are covalently bound to the surface via an epoxy group.

112. The substrate of claim 85, wherein the selectin molecules are covalently bound to the surface via a group selected from the group consisting of amine groups, aldehyde groups, and combinations thereof.

113. The substrate of claim 85, wherein the selectin molecules are covalently bound to the surface via a group selected from the group consisting of vinyl groups, thiol groups, carboxylate groups, and hydroxyl groups.

114. The substrate of claim 85, wherein the selectin molecules are covalently bound to the surface via a linker moiety.

115. The substrate of claim 114, wherein the linker moiety is covalently bound to the selectin and to the surface.

116. The substrate of claim 114, wherein the linker moiety is non-covalently bound to the selectin and covalently bound to the surface.

117. The substrate of claim 116, wherein the linker moiety is bound to selectin via a non-covalent ligand/receptor pair interaction.

118. The substrate of claim 114, wherein the linker moiety comprises one or more moieties selected from the group consisting of dextrans, dendrimers, polyethylene glycol, poly(L-lysine), poly(L-glutamic acid), poly(D-lysine), poly(D-glutamic acid), polyvinyl alcohol, polyethylenimine, and combinations thereof.

119. The substrate of claim 85, wherein the substrate further comprises one or more cell modifying ligands.

120. The substrate of claim 119, wherein the cell modifying ligand is targeted to a specific cell type.

121. The substrate of claim 120, wherein the specific cell type is a cancer cell.

122. The substrate of claim 120, wherein the specific cell type is a stem cell.

123. The substrate of claim 119, wherein the cell modifying ligands are bound to the surface through a covalent bond.

124. The substrate of claim 119, wherein the cell modifying ligands comprise at least one cell modifying ligand that is covalently attached to the surface and at least one cell modifying ligand that is non-covalently attached to the surface.

125. The substrate of claim 119, wherein the cell modifying ligands disrupt cellular function in cancer cells.

126. The substrate of claim 121, wherein the cell modifying ligands comprise tumor necrosis factor (TNF)-related apoptosis inducing ligand (TRAIL).

127. The substrate of claim 122, wherein the cell modifying ligands comprise ligands selected from the group consisting of basic fibroblast growth factor 2 (FGF-2), bone morphogenic protein 2 (BMP-2), and combinations thereof.

128. The substrate of claim 119, wherein the cell modifying ligands disrupt or induce one or more processes selected from the group consisting of cell quiescence, cell proliferation, cell migration, cell de-differentiation, cell spreading, cell attachment, and cell differentiation.

129. The substrate of claim 85, wherein the substrate is an intravascular stent.

130. The substrate of claim 85, wherein the substrate is a vascular graft.

131. The substrate of claim 85, wherein the substrate comprises a glass.

132. The substrate of claim 85, wherein the substrate comprises an implantable and/or injectable material.

133. The substrate of claim 85, wherein the substrate comprises an injectable polymer.

134. The substrate of claim 132, wherein the implantable and/or injectable material stimulates cells to produce a cell modifying ligand.

135. The substrate of claim 132, wherein the substrate is implanted into a site that serves as a niche environment for stimulating vascularization.

136. The substrate of claim 85, wherein the substrate comprises a porous polymeric matrix and the surface comprises the surface of the pores.

137. The substrate of claim 109, wherein P-selectin is linked to the surface via its C-terminus.

138. The substrate of claim 109, wherein P-selectin is linked to the surface via a cysteine residue in the intracellular domain of P-selectin.

139. The substrate of claim 85, wherein the substrate is substantially degradable.

140. The substrate of claim 139, wherein the substantially degradable substrate comprises at least one surface-erodible polymer.

141. The substrate of claim 140, wherein the surface erodible polymer is selected from the group consisting of poly(glycerol sebacic acid), polyanhydrides, poly(diol citrates), and combinations thereof.

142. The substrate of claim 139, wherein the substrate comprises a hydrogel material.

143. The substrate of claim 142, wherein the hydrogel material is selected from the group consisting of poly(ethylene glycol), hyaluronic acid, and combinations thereof.

144. The substrate of claim 139, wherein the substrate comprises one or more cell modifying ligands.

145. The substrate of claim 144, wherein the cell modifying ligands are exposed as the substrate degrades.

146. The substrate of claim 85, wherein comprises entrapped cell modifying ligands.

147. The substrate of claim 146, wherein the entrapped cell modifying ligands are enclosed within releasing vehicles.

148. The substrate of claim 147, wherein the releasing vehicles are selected from the group consisting of nanoparticles, microparticles, and combinations thereof.

149. The substrate of claim 147, wherein cell modifying ligands released from the releasing vehicles are transported to the surface of the substrate.

150. The substrate of claim 139, wherein the substrate further comprises particles or regions of more slowly degrading materials that contain entrapped and/or surface-bound cell modifying ligands.

151. The substrate of claim 150, wherein particles or regions containing cell modifying ligands are exposed as the substrate degrades.

152. The substrate of claim 85, wherein the cell rolls over the ordered layer but does not stop.

153. The substrate of claim 85, wherein the substrate further comprises antibodies.

154. The substrate of claim 153, wherein the antibodies facilitate stopping cells that roll over the ordered layer.

155. The substrate of claim 85, wherein the cell invades the substrate and becomes entrapped.

156. The substrate of claim 155, wherein the entrapped cell is a circulating cell.

157. The substrate of claim 156, wherein the circulating cell is selected from the group consisting of metastasizing cancer cells, stem cells, progenitor cells, and combinations thereof.

158. The substrate of claim 156, wherein the circulating cell is an endothelial progenitor cell.

159. The substrate of claim 85, wherein the substrate comprises a prefabricated vascularized matrix.

160. The substrate of claim 159, wherein the vascularized matrix is implantable.

161. The substrate of claim 160, wherein the vascularized matrix is created with endothelial cells from a patient and the substrate is administered to the patient.

162. A stent comprising a substrate of claim 85.

163. The stent of claim 162, wherein cell rolling over the ordered layer of selectin molecules facilitates separation of cells into subpopulations.

164. The stent of claim 163, wherein the subpopulations of cells can be quantitated.

165. The stent of claim 162, wherein cell rolling over the ordered layer of selectin molecules facilitates collection of cells from a sample.

166. The stent of claim 165, wherein the sample comprises a blood sample.

167. The stent of claim 162, wherein the stent can be implanted into the vasculature.

168. The stent of claim 162, further comprising cell modifying ligands that facilitate delivering apoptotic signals to cancer cells.

169. The stent of claim 169, wherein the apoptotic signals are delivered to cancer cells before they metastasize.

170. A vascular graft comprising a substrate of claim 85.

171. The vascular graft of claim 170, wherein cell rolling over the ordered layer of selectin molecules facilitates separation of cells into subpopulations.

172. The vascular graft of claim 171, wherein the subpopulations of cells can be quantitated.

173. The vascular graft of claim 170, wherein cell rolling over the ordered layer of selectin molecules facilitates collection of cells from a sample.

174. The vascular graft of claim 171, wherein the sample comprises a blood sample.

175. The vascular graft of claim 170, further comprising cell modifying ligands that facilitate delivering apoptotic signals to cancer cells.

176. The vascular graft of claim 175, wherein the apoptotic signals are delivered to cancer cells before they metastasize.
